SEQUENCE LISTING (35 sequences)

SEQ ID NO: 1 - DNA, 1611 bp, Glycine max
atggcgggaggacaactaggagtgcttaatgcactcgatgtggcaaagacacaatggtaccacttcacagctattgtgattgctggaatgggattcttcaccgatgcctatgatcttttctgcatttcccttgtgaccaagttgttggggaggttatattacacagacataaggaacccgaagcctggtgttcttcctcctaatgttcaagctgctgtgactggtgttgcactatgtggcactttagccggccaacttttctttggatggcttggtgacaagttgggaaggaaaagggtttatggcttaactcttatgcttatggttctgtgttccattgcctcgggactctcttttggcgacacccctaagggtgtcatggccacactttgtttcttcagattctggcttggctttgggattggtggtgactaccctctatcagctacaattatgtctgaatatgccaacaaaaagactagagggtcattcattgctgcggtgtttgcaatgcagggttttggaatcatggctggtggaatagttgccttgattgtgtcatctgcatatgaccacaagtacgatcttcctagttacaaggacaatccagctggatcgaaagtggattcgcttgattacgtttggcgtatcatcttgatgtttggtgcagtcccggctgctcttacctactattggcgaatgaaaatgccagagacggctcgctacacggcccttgtagccaagaatgcaaaacaagctgcttcagacatgtctaaggtgttgcaagttgaggttgaagctgaagaggacaagttgcagcacatggttgagagtgaaaaccaaaagtatggcttgttcagcaaggaattcgccaaacgccacgggctgcacttggttggaaccacggtaacttggttcttgttggacattgccttctacagccagaaccttttccaaaaggacattttcactgccattggatggattcctcctgcacaagacatgaatgcaatccatgaagtttataggattgcaagagcacagacactgatagcattgtgcagcaccgtgccagggtactggtttacggtggcctttatcgatatcattggacgtttcgccatccaattgatgggtttcttcttcatgacagtcttcatgtttgctcttgctataccttacaatcactggaagaatcacaacaatattgggtttgttgtgatgtattctttcacctttttcttctccaattttggtccaaatgccaccacatttgttgtgccggcagagattttcccagcaaggctgagatctacttgtcatggaatctcagctgcggcaggaaaggcaggagcaattgttggtgcatttggattcttgtatgctgcacaaagtacgaaccccaacaaggttgatcatggttacccaactggtattggggttaagaactccctcattgtgcttggtgtgatcaacttctttggaatggtattcaccttgttagtaccagaatcaaagggaaaatcactggaagagttgagtggggagaatgaagatgatggtgctgaggctatagagatggcagggtctgctaggacggttccagtttaa

SEQ ID NO: 2 - DNA, 1611 bp, Glycine max; identical to SEQ ID NO: 1

SEQ ID NO: 3 - protein, 536 aa, Glycine max
MAGGQLGVLNALDVAKTQWYHFTAIVIAGMGFFTDAYDLFCISLVTKLLGRLYYTDIRNPKPGVLPPNVQAAVTGVALCGTLAGQLFFGWLGDKLGRKRVYGLTLMLMVLCSIASGLSFGDTPKGVMATLCFFRFWLGFGIGGDYPLSATIMSEYANKKTRGSFIAAVFAMQGFGIMAGGIVALIVSSAYDHKYDLPSYKDNPAGSKVDSLDYVWRIILMFGAVPAALTYYWRMKMPETARYTALVAKNAKQAASDMSKVLQVEVEAEEDKLQHMVESENQKYGLFSKEFAKRHGLHLVGTTVTWFLLDIAFYSQNLFQKDIFTAIGWIPPAQDMNAIHEVYRIARAQTLIALCSTVPGYWFTVAFIDIIGRFAIQLMGFFFMTVFMFALAIPYNHWKNHNNIGFVVMYSFTFFFSNFGPNATTFVVPAEIFPARLRSTCHGISAAAGKAGAIVGAFGFLYAAQSTNPNKVDHGYPTGIGVKNSLIVLGVINFFGMVFTLLVPESKGKSLEELSGENEDDGAEAIEMAGSARTVPV

SEQ ID NO: 4 - DNA, 7069 bp, Glycine max
tatttaattttaaacaactcattaaatgacttgattcccctatttcctcgttggtacggcaattaagatgtacatctaatccggtcggaacattaaatagtatgacctacgtgtgtgattgattgcctgcagtacaattaacagtcatagaaaatctgtgaagcctataagcttgatgcttactccgtctaatttgactggaaatatatatagaaagaaaaaaaaaaggaaatagagaggatagggattgaataaaaaaattactttcttaatatttatttaagagaaaaaatattttatattttctaagttatgataaaattggagataaatgtttattgttatattctaccaccattctatttatttaaataagtagagaaaaaaatatccacttatttatttcttatttttatatattccaaaaatacatttttgtaatacatttttattttatttctatcactcctattttcttcttcttatttttttatttcacttctaattcaaaaaaatattattgtttataattcctttatgttattttttatcacttttttaagttgtaatttaaagttatagaatataacttaagagatataattggtatttttattcatattaaaactaatcaaatttttaatatatttttatatcttttattcattattcaaacatttcatatattttttttactgcagtttcatataatattattattattactattatttcttttaataagtataaatacaagagtataattaataaaataataattattactatcttgaactttttttagagcatactattggatttttttacctaaaaaatacaaaaaaaaatccacaaataattctcacgtttttttttccttagttaagaaatcagttcatggtacattgagaaacttgattctcaaggaatcaattctcaacatgcttttttttaatctaagaatcgatgtgtttgatttttaaaaaaaatcttttgttatttatttttaatattttgtttgtatatatttaaatttatatatatatatatatatatatatatatatatatatatatattagaactaacaaatgttttttggcatatgttgttgttagttttatattgatttttttgtttaaaattatttatcatgttttcctttctatgacacaacatgcaccttgtatctccactattgttatggaaataaatttccaagactaagatgattttcaattaatttatttaaaagaaaacactcataattcattttttatcaaagaatatttttttaagaaacaattattcatcaaatgaaatattaagaataggtgacataaattgaaagtaaacatacctaaaaaaagaaaaagaaaaagatgaaactaagtttatctagattgaagaagaaaaaaaaaataaaaagataaataactcaaagactagattaaagtacataaaaaagttagaacaactaatttatgcaatgactatacagatttattacgcatcagagtaatgatctcaacaacagttattggttttcttggtttttgcatcaaaacaaggatttcatttggaaaacgcctaaaagttgtattcaactcaattgaacctttggtactaaattccgaataaatgaaaaaaaaaattctcacatcattgtcgcatttgagctccatataattgtattcaacacaatcatctatgtaaattggttgacgataaaaaagatcaagtaaaattctacaacctctgttgacgtcaacaattatttttcttacggcatcaaacgacatgttctcactattagcttagtggtagagtgtgtccctttgtatgatggaatcatgtcaccattacaatacataacaatggatgcactctccatgttttaagcaatatggtgaatatcaagtaaaggtgttgaggttcatttaacccctactactatttgtattaggtgataaactcaaaagcagtttagattgtgtcttgatttattatagttggtttgcgaattaaaaaaaaatagtgttcaatcaaatcatctcataaaaagtattgttcactcaaattgtctcgaaaaaagcattatgaggtcaaatcatcttgataaaagcattgttcaaaaaattcgtctcagaaaatgcaatgcttaatgaattcgtctaagaaaaatgcagtgctcaatcaataaatacaatgttcaatgttcaatcaattaataagaacaaactgacattcatagtacaccataagttaacaagggtaattaatgttgtctgtgttcactagttccacatatgtgaggttgcatgtttcttgttggttgtccgcaaactcgcccatgttcttatgattgaagttatttattaggaacaacagtttgttcttccaaagaaatcattaaaacatcattgattttaatctatccgagttttgcaacatctgaaaacccaacctaaacctacaaagattcaattctaggatctagagaagacatatcttgagcatatgactggattacatcatttgtagaaatcaaaataataacaacatgactatttttaaatcataaaataattaatattcaattaaacataacgtattacatatttctccaggtctatgaaggtgttgtttccaaagaaggtgaatgattgtagctttcatggtttaggctttggtactaatgttaaaaagaagttaaagagtgatatacgagagaacgagagaaagtgagtcggtgtgcatatgatgacattgcatgcattgtatcttgagtgttaattttgtcaatgggatcttgatcattgaacatgtagtgctcaaatcggtgttatctaagccgatgattttttcggtgggatcttggtcattaaacatgcgttgctctacccagtgttatctaaatagttgattttgacagtgtgatcttggtcattaaacatgcgttgattcaatcaattttatctaagtcgttgattttatcagtgagattttgaccgttctttttaatgtgcatgtgataagtcattaaacattataaatataatatgcaatgaggtagtctaatacatcacctcaatcaaaaaccttactgctaccctaaacctaaaccatgacttcattatcaatctcgaaagttgtcttatcgatttcattaacatgaaaactatgggaaagaaacattatttgaaccaaaataactgatacatacaacaacctaggtgtcgaaagttgtcctcatgaattgaaattttggaattttagtgatttttcttgtttttgaccactttgttaatgtgtttcaaggtcaattggagtcccaaaagttgtcttattgacttcattaacatgaaaactatgaaaatgcaacattatttgaatcaaaataactgatacacataataatctagatgcaaaaagttatcctcatggaatgaatttttttagataaaaatgattttttcttgttatgaaaatttgcatgcacatttctgttaattagtttaatacatgaatatgataaacaatgaaacctaacctaaaccctaattatgtaccatttttactaagtatgaccatgaaccctatccctaattctggacaccaaaccctaaatatattaactaaaccctaggtttgaaaccttaccctaaccataaccctaattatggatactaaaccctaaatgtatcaaagacacatttgttttacatcaatgcgttagggacacttttgatttaagctagatgaagggtggtgtgactatgacaaattctaaaaattgcacatgccttgtctcatgttactgctgcttgtaagcatgttcaccatcagtataggaactacatacatcatgtgtatacattgaaaagtgtctctaatgtgtacagaagattgtttgttgaattgcgcaacgaaacatattgaccactatctcatgagccaagaatctgccccgacccgaagatgaaaagaaattctaaaggtcgtcctgtctcctctcgtatacataccaaaatggatatccaagaattggattagccaaaaagatattctatgtgtcgcatcctaagccatttaaaaaataaatgtctttaccgtgtaggctcacgccaacaacgttaagtttatgtcatattgtttacaatttaaaaattttgttgtgagaaagagaagaagacaacaaaagaaaatagtttttttcttgggtcaaacatcgtggttgtccctcaacctattgtgtttgataccaatctcatttattatgtgtgactagctacatcaacaaaattacaaactaaaatatatgaataaagtttagaaaaatttaaaactaacatttcatcttataattagtttttcatctcaatcataaattatcatatcccaacaagttattagtctaagaaaaaaacataattgtgaagggagaccacaataaatatgggcaaccaagcttttttttttttttttacacggattaatcacaaagaaaattggtgaatactccacactaatatcatagtagttaaaaaaatcataaattatcatatatgttaaatttgttggattttgtaatatctatcattaataataataataataattttcatttaagaatgagagggaaaatattaattattaaattttattaaatgatcaaaattaaaaaaatatttttttattaaattttcattcaaatttcgttccaatattctatatttttttattgtcatgtaaaaatattctctgttttatattaatatatacaccaaattacaaaaatgattacatactctgtctgttttaacttttaatattttaaagcaaagcaagcaattgttaaaaagaacgtaaagaaatagatgaagcaagcaatgttatatatatatatatatatatatatatatatatataacaatagcttattctttcggttttaactttcaatctttaatgtcattaaaaaaaaaaaacttataatctttaaaagcaagcggccaaagcattagaaagagcgtaaagagatagatgaagcaagcaagcgatttgtgaaagcgttgaccagaaagtgtgaactacaaagtgtaggaatgagcatataccaccagatacctgcgttgattctggattcttgaagccattccttctattttctggtgacagcttcgtcttgtgctatactatataacaactcaccctctctccttacctcttcattcccaacacaaaacactctctcctccttcaacctagccctggtcactaccaaccaagtgtcccccctcctctcccttgttaacaggtgagacccttttttcattttccttaatggaactttttggttatgcatggcatggaagtcaaactaactcactggtagttgtagcctctggttgagttccttggtgggaggcccacttttctaagagcacaaacacaacttcacttcccttttccaattttataatttactaatcattaccattccaccgctaaaaatctggaacttgctttttacccgtatgatatgatacgaggagaccaatttcgtgattcttctcctctcctcatcatccaccactgacccactcaccctttttgcaaaatagtggttgtgttatgttatccatattccatacccatgaagtgaaacacaaaagaaacaacttttagccttacccatcctttttttcctacagtttatgctgattttccatgaaacaactccaaccccgtccctctaaacaaaaaccgaaccttctcaacggaattaattgttcaggaaattattggcacaaacacaaaccattctcctctgccgtttgttttttaaatttattttatttttataatttaggatgataaatcttttccagtgtctatgctttattttgtcttttaccaaattttgagttttttctggtttcctcgtgctacccatggtctgctagcgacttggttcaacacctgtagattccctgtttgcagttgattttccacaccctttttttcttcacgtctctgttcttttggtttggtggggacaccagaaaaagaaaaatagtaccaaattaccaatgaatttccaatttctgaaccaaattttctctatcattgtatatactgaagttgactcattggttgaatccacaccttaagcaatttgggtgaagtggttgaaaagtgagaatagatctcaccttcttttactaaattctcacacgactttgatatgagaaaattgtgttgatctttcttttcatacataaaatcttgtctatttaataataaacacaattttatatgacatggctatgtgaaaatatctattacatgacaagctttccggagctggaattatagagtcgttgttagttcagagtcaaattcatataatatattaatgtaatagttttattataggaattttcaactttggtggctgttcactatgaatatattagaaaaataccaaaaagtaagggtttttcatcaatccaatgcttcagcctgagaaagatgaacatctgccgtatttttttttcctttcgaaccttcattaaaattcttaatttcttacaaaagtctactaaaatatacacttaatttgtcgattattcaatattttattcttgataattagtacaaagtgtggaactcgttatatcattccttcactgttattgcaaacaccagttcttttccattcaaattgttagtattgaagagggcgagccctggtgcagcggtaaagttgtgccttggtgacttgttggtcatgggttcgaatccggaaacagcctctttgcatatgcaaaggtaaggctgcgtacaacatccctcccccataccttcgcatagcgaagagcctctgggcaatggggtacgaagggaagaaaaatatggaattgcttgcttatagtcttagactatagttgaatttgacacatacaaaaactagtatctttttttttatttttttatttttgtttcttgtgctggaaaattctaatcattgattgtggcacatcaggtagctgagttagtgagtg

SEQ ID NO: 5 - DNA, 1566 bp, Glycine max
atggggaaggagcaagttcaggtgctgaatgccctggacgtggcaaagacacaatggtatcatttcacagcaatcatcattgctggaatgggcttcttcactgatgcttatgatctgttctgtatatcactagtcacaaagctacttggtcgcatttactaccacgttgatggggctgcaaagcctggttcattgcctcccaatgtgtcagctgcagttaatggtgtagcttttgttggaacactttcagggcaactcttctttggctggctcggcgacaaaatgggccgaaaaaaggtctatggcatgacccttgcgcttatggttatagcttccattgcttcgggtctatccttcggacacgatgcaaagactgtgatgacaactctatgcttctttcgcttctggcttggttttggcattggtggagactaccctctttcggccaccataatgtctgagtattctaataagaagactcgaggtgcctttatagctgcagtgtttgccatgcagggttttggaattttggcaggaggtgtgtttgctattatcatagcatctgtgttcaagtccaagtttgattctccaccatacgaggttgatccgttgggttcgactgttccacaagcagactatgtttggaggataattctcatgtttggagcaattcctgctgcaatgacttactactcgcgatccaagatgccagaaaccgctcgttacactgccttggttgccaagaatatggagaaggctgcagcagatatgtctaaggttatgaacatggagattcaagccgagccaaagaaggaggaggaggcacaagctaaatcatatggattgttctccaaggagttcatgagtcgccatggactgcatctgctcggaacaacaagcacatggttcttgcttgacattgcattctacagccaaaatcttttccagaaggatatcttcagcgcaattggttggattcctccggcaaaaacaatgaatgctcttgaggaggttttctttattgcaagggctcaaactcttattgctctatgcagtacagttcctggatactggttcactgtggccttcattgataggataggaagattcgccatccaattgatgggattcttctttatgactatcttcatgtttgctcttgccattccctatgatcactggactcttagggagaacagaattggatttgtggtcatttactctctcacattcttctttgcaaactttgggcctaatgccaccacatttgttgtgccggcggagattttcccagctagatttagatccacttgccatggaatatcttcagcatctgggaagctcggggctatggttggtgcattcgggtttttatatttggcacagaatcaggacccgagcaaagcagatgcagggtaccctgcaggtattggtgtgaggaattcactgcttgtgttgggtgtgattaacattttaggcttcatgttcactttcttggtgcctgaggccaagggtagatccttggaggagatttcgggagagcaagaagaggagaccaaggtgtaa

SEQ ID NO: 6 - DNA, 1566 bp, Glycine max; identical to SEQ ID NO: 5

SEQ ID NO: 7 - protein, 521 aa, Glycine max
MGKEQVQVLNALDVAKTQWYHFTAIIIAGMGFFTDAYDLFCISLVTKLLGRIYYHVDGAAKPGSLPPNVSAAVNGVAFVGTLSGQLFFGWLGDKMGRKKVYGMTLALMVIASIASGLSFGHDAKTVMTTLCFFRFWLGFGIGGDYPLSATIMSEYSNKKTRGAFIAAVFAMQGFGILAGGVFAIIIASVFKSKFDSPPYEVDPLGSTVPQADYVWRIILMFGAIPAAMTYYSRSKMPETARYTALVAKNMEKAAADMSKVMNMEIQAEPKKEEEAQAKSYGLFSKEFMSRHGLHLLGTTSTWFLLDIAFYSQNLFQKDIFSAIGWIPPAKTMNALEEVFFIARAQTLIALCSTVPGYWFTVAFIDRIGRFAIQLMGFFFMTIFMFALAIPYDHWTLRENRIGFVVIYSLTFFFANFGPNATTFVVPAEIFPARFRSTCHGISSASGKLGAMVGAFGFLYLAQNQDPSKADAGYPAGIGVRNSLLVLGVINILGFMFTFLVPEAKGRSLEEISGEQEEETKV

SEQ ID NO: 8 - DNA, 5914 bp, Glycine max
aatccttggttggaccttgtttgtcaacccctgatacgtaataaccatcattgatcatcaaattgcataatcgcgttggaaagtgttagcctattaaggcctaataaggcctcgtttggtctcagccctcaaatattgaattgaatgttggtggattgcttcaattctgtgtatgtgtctatgaatgaacgaaaaattggaaagcttcccactttagctatgaggtatctaaatctttgggctctcaatctcatgctaccacgggccagtcaagtgcatcatatgcaagttctaatccataaccacgacaaagtccaaaagctatatggcataaactaaaatccatgtacattttcagtcgtaaatgtacattgtatttgtattagttgttacataattataaaataaaaggttgtactctgtcacttttctatttagagtgtgttaaggaagttattttaacaagtttttaattttttttattaattgaaaaatttgtttggttatttgataattgattttttttaatagtttctagggttttttgaaacgttaacttaaaatattattttttgaaatttacttcaactagtgttttttaaaatgttaatctctaatttttagtttgttatatttatttttttccttgaatttttattatatttccttttcaatcttttctatttaaggtaaaattttattgttattttttctttatgtaattttatattttctaactatttcaataaataatattatcaaacacttatgattcaataagataattttttaattttcaattaacttttagctatcggttagcttttcaactttcaagtaatttttcatgtagttctatcaaatatagcattaatgtattgctttttcttattgaaaaataataaaataattattttaaggctttttaattactttatttatgataaaaaaattcaaacaagtgttttaattaaaaattatcttaacaaaacactaaatgggtcttaataagttgttgtagaaatttaactattttaaataatcattttttttcttgtgtataatgatactactagtattttagttacaacttcaactttatgtaaactactcaaatctctcactactaagttgatcctagtatgttttaattactttcttctattgatgaatgaactgtaatacgagtgttgacatataattttgattttgatttaaattcaaaatcattagaattacgagtttgtttttcttagctaattatatttcattagactaaaatataaattattataattttagattttcattctcgcatattattttcagttatatatataacaaaaaaatgcttaatttattaagaacaataaaaatagttaattttaattaatatcatcataaatttaaattttatttcaaaaatactcataaaatttatatttttaataactcaataaatgtattcacatacattcatggataatgagtgatatgtaattagtagacttcacattgaatcaatcatataaaaagaaactatcaattaatatatttaaaagtaataatatatttcttataactaggatatttttttataatttatttcttatactacatttattacatatgaagcagaaaaaaatcatttattatacaaaatttcaaatttaataaaactattgactataaaaaggttttttctttaataattctagacaacaattaattcaataaatttcaagcattaatattacttttaatttatgcataatttttctataattttaaatttatgcattttattattatctttatccataatatcattattaaaaaaattattattaaatataataaaaaatacttattattattatacaagatgattaaaataaaagaaaagggaaaatagaagaaggatctaaataccatagattaaaatacataaatttcaaagtttaaaaagaaaataaaaaggaagatcagtttaattgggttggaaaaagaaaaatatgagaaccaaattcattgcaattaatttgagtttgaagtaaaataaactaaataattcaatcaattcaaattgatttagtttaaattattatcttgattaagtgaattaaatccataaacaccttaaagtgcattataaaaatcgtcactaaggcactaaacttttttattttcttgatttgccagatttccaatgtagagaagacggacttgttaagaaaaatatgcctttttcttctgagattcattcgatttttaccgtaatggtatccacgttcttaaggaaaagagttgaaattgaaactctatgagaagcaaccaaggtgtggtaccctccgtgcatactttgggattgttaccttaacttaaatgcacgttctaattgtgtaataattagacaagcgtcttaacttaaatgcacgttctaattgtgtaataattagacaagcttacttgttacatttattttaacattttgatataaatcttcttatattagagagattttttattttctctagtaaccgaaaccaaatatgtcccgtttattgttgtatcattttgagtttaatataatttaaatttttctcaaaaaattaacatgccttccaaaatttgactgtaacttttatttaacttatataacaagtttccaactggcaactacactcaaaagcaaaagactttctcgaaatttctgggtgtccaaaaccgaagatgaatggctggttttggagaggtgctactaaacccgatatacttttttttaatcatgatcttaaagatatagagattaaaacatacgaaatacatcattttaattactaaaaataataaatattaaatgacatttacatttttcatacaataaatgcgtctttctttagtaattactttctacttttgttaacatctgttttttttttcttttaataaataagattgttattgatgaaggttcgcaattcaaaactcaagtgcctcatctactaactaacataaagcgatataatccgattgataaataattatgttgcttaaatattagaaattttatttctgagtcgatgtatataaaaaaatatttattagaagatattaatctcataaataaattttcatatattaaaagattaattttttactttcaattagtaatcgacttggtataaaaaaaaaagcctaattcaggttcatcgctgttactctttttaaaatctctattttgctctttttgagatcttaggggttacagatacctgcaaatgactttcagaaatttgggaatcttctctttaccacatgtaatatatgaagggccatttaagaaaaggtataaatgttatattataagataggggcaaccaagattatggcaatatagatttggcactgttggaaattggaatagtgtcacctcctaaataaagctggagtaaagaatgttgaaggctacctagtacctgcaatggtttatctttgaccaaagatctagtgggggttgccacatataagaattgtagaagaatatcacacccggaatatacaagtacacggtaaagtagtcttgatcttggaccgaaaataatgagaacaagggccccataagtttgattccaaattgaaaccacatcatggaaaagtcacttgtcctacaacttcccatgccatgtggaaagtggatggcaacatttataattactacgatgatgactgcttggtgagtcatccaagtttgaatgtaaattgaactgaagtcagtaaaatttggaaattttggattataagggacccaaaacttgattacaaattggagccaactttgagatacttacattttccgttggtcgtggtttgtccatttgaaagtggtgggcaataatgatagtttctattatgattttttagtaaaagttgtagaatcgggagacacttctaatactgctagatagggttaatgacatactgaaactatcaacaaataccacttaaaatattactgcattatgcaaagattgaaaccataaacagttccacctggttgtagtttatattcttttatgatctatgaagaaaacaacaaggatccaacttacttttcagcaaaaatagaattcatttctcataatttagttcttccctttatcttattgactaatttgatttttaacccttctttgactaggttaattaaatgggtttgttgtttcataccaatggccacaaagaaaagttcataaaacaagctgtgaattgtgatctcatccagtgtaatataataggcaatgctgatgacatgtacaaacaataagtggctaccaacagtaataaataaacagcatagagagattccaaaaggggacttaaaagccataaaaaatattttgttttattgcttgaagaagaggcaaaccaaaagcttgaatggattgtcttgctgctatgattgtgtgcatatctgagaaggagatcactgccatgtttagtaaaattcattaaagagtgttagaggaattttgattaatctataaagccggctagggaaagaaaaatgactgttggatataagttcaagctattgtattgactgctactttttttttttaatatataaaatagagagcttcacttcttgatgatgtgatgactaatcactctttaatttcaaaaggaaagttgaattggcccctagatattatgaaaagaagacacatgatagggatcaattaacaactaagctaaacagtacagtatggctacatacgcggcccagattatttttagctctgaaatcaaaatcatggtattttttgaaacattatgatatataattaaaaagaagaagacaaagtggtttgctggcatggctggtgagagagaacgaaattagtggagagtaaggaatttaaaataatttatcatcgatgttaaattcttttagcaaatcctcgtaacatatttctaattatttcaaacagaaagtgtaaacgtgtttaatatcttggagagggttatttaattggttcattaagaatatataaaactatttttgaaagcaaagtgggctttgagtctcagcaagtggattcactgctgcaataagagcctccttctatcaaaagtcaagcacgaatgaaaaagtaaagtgtgggtgcccatgttttaaagtttatatcgacactttgacattcatatcctttgtatttcaattagattagatatgttctacggacattatttgatctttaaattccctcaaatgcctgtggtgagctaatataatatgtagtgtagtgtatgtatgtatgcggtaaaaaaataagctgaaatttggcgaggacaatatacaagtcccaaatttaatatgaatatattataaactaagtactgcttaaatagtagaaaaacagaatgagataatcaaattaggtcagggatctcaaaacttcattcccatgtaaaccagtagaagaaaaataagtgtgtaagatacaattaggtcaggtctatgctggttagagttagatttaagattcaagaaaaagttaggccggtaattaggttaaggtttagattcaataaaccaaataaatgttttttttaatccatttaattattttagtgagcacagtacaaatttctttcttgagtttcaacttttgatggttttttaaaatgaaaaagtaataagcaaatggataatgaaaagatgatgatagcacttcttagttctcaatcatcaactatttaaaacaatggtcagaggctaacttctccactagtttttctgtgtagagccctttggacacaccctctaaaccaatctatcaagtcctgaatctggtgagcaaatatggagctttgttggcaaacatccatccatggttcttgaagaagc

SEQ ID NO: 9 - DNA, 21 bp, Artificial Sequence
  GmPT7 fwd primer
atggcgggaggacaactagga

SEQ ID NO: 10 - DNA, 23 bp, Artificial Sequence
  GmPT7 rev primer
ttaaactggaaccgtcctagcag

SEQ ID NO: 11 - DNA, 33 bp, Artificial Sequence
  GmPT7 fwd primer 2
atcggcggccgcatggcgggaggacaactagga

SEQ ID NO: 12 - DNA, 33 bp, Artificial Sequence
  GmPT7 rev primer 2
atcgggatccttaaactggaaccgtcctagcag

SEQ ID NO: 13 - DNA, 31 bp, Artificial Sequence
  GmPT7 fwd primer 3
atcgtctagagatggcgggggacaactagga

SEQ ID NO: 14 - DNA, 34 bp, Artificial Sequence
  GmPT7 rev primer 3
atcgggatcccgaactggaaccgtcctagcagac

SEQ ID NO: 15 - DNA, 32 bp, Artificial Sequence
  GmPT7 promoter fwd primer
atcgctgcagccttgtctcatgttactgctgc

SEQ ID NO: 16 - DNA, 33 bp, Artificial Sequence
  GmPT7 promoter rev primer
atcgccatggccatcactcactaactcagctac

SEQ ID NO: 17 - protein, 15 aa, Artificial Sequence
  Synthetic peptide
EDKLQHMVESENQKY

SEQ ID NO: 18 - DNA, 32 bp, Artificial Sequence
  GmPT7 fwd primer 4
atcgggatcctatggcgggaggacaactagga

SEQ ID NO: 19 - DNA, 31 bp, Artificial Sequence
  GmPT7 rev primer 4
cgacgcgtttaaactggaaccgtcctagcag

SEQ ID NO: 20 - DNA, 32 bp, Artificial Sequence
  GmPT7 fwd primer 5
catgggatccggctgctcttacctactattgg

SEQ ID NO: 21 - DNA, 31 bp, Artificial Sequence
  GmPT7 rev primer 5
catgaagcttaaggccaccgtaaaccagtac

SEQ ID NO: 22 - DNA, 31 bp, Artificial Sequence
  GmPT7 fwd primer 6
catgctgcagaaggccaccgtaaaccagtac

SEQ ID NO: 23 - DNA, 32 bp, Artificial Sequence
  GmPT7 rev primer 6
catgacgcgtggctgctcttacctactattgg

SEQ ID NO: 24 - DNA, 33 bp, Artificial Sequence
  GmPT7 fwd primer 7
gagctgagctcatggcgggaggacaactaggag

SEQ ID NO: 25 - DNA, 34 bp, Artificial Sequence
  GmPT7 rev primer 7
ctagatctagattaaactggaaccgcctagcaga

SEQ ID NO: 26 - DNA, 40 bp, Artificial Sequence
  GmPT7 fwd primer 8
tcaatctagaggcgcgccggctgctcttacctactattgg

SEQ ID NO: 27 - DNA, 39 bp, Artificial Sequence
  GmPT7 rev primer 8
tgccggatccatttaaataaggccaccgtaaaccagtac

SEQ ID NO: 28 - DNA, 32 bp, Artificial Sequence
  GmPT7 fwd primer 9
atcgagatctgaacatggagattcaagccgag

SEQ ID NO: 29 - DNA, 33 bp, Artificial Sequence
  GmPT7 rev primer 9
atcggaattcccagtgatcatagggaatggcaa

SEQ ID NO: 30 - DNA, 33 bp, Artificial Sequence
  GmPT7 fwd primer 10
atcgctgcagccagtgatcatagggaatggcaa

SEQ ID NO: 31 - DNA, 32 bp, Artificial Sequence
  GmPT7 rev primer 10
atcgtctagagaacatggagattcaagccgag

SEQ ID NO: 32 - DNA, 32 bp, Artificial Sequence
  GmPT7 fwd primer 11
atcgccatggggctgctcttacctactattgg

SEQ ID NO: 33 - DNA, 31 bp, Artificial Sequence
  GmPT7 rev primer 11
atcgagatctaaggccaccgtaaaccagtac

SEQ ID NO: 34 - DNA, 31 bp, Artificial Sequence
  GmPT7 fwd primer 12
atcgctgcagaaggccaccgtaaaccagtac

SEQ ID NO: 35 - DNA, 32 bp, Artificial Sequence
  GmPT7 rev primer 12
atcgtctagaggctgctcttacctactattgg

(19) United States
(12) Patent Application Publication
(10) Pub. No.: US 2019/0367937 A1
(43) Pub. Date: Dec. 5, 2019

(54) EXPRESSION OF A PHOSPHATE TRANSPORTER FOR IMPROVING PLANT YIELD

(71) Applicant: Fujian Agriculture and Forestry University, Fuzhou City, Fujian (CN)

(72) Inventors: Hong Liao, Fuzhou City, Fujian (CN); Liyu Chen, Fuzhou City, Fujian (CN)

(21) Appl. No.: 16/484,571

(22) PCT Filed: Feb. 8, 2018
(86) PCT No.: PCT/GB2018/050361

§ 371 Date: Aug. 8, 2019

(30) Foreign Application Priority Data
Feb. 9, 2017 (CN) PCT/CN2017/073155

(51) Int. Cl. C12N 15/82; C07K 14/415; C12N 15/90

(52) U.S. Cl. CPC C12N 15/8261; C12N 15/90; C12N 15/8213; C07K 14/415

(57) ABSTRACT

The invention relates to a method of increasing yield in plants comprising increasing the expression of a nucleic acid encoding a phosphate transporter (PT7) polypeptide. The invention also relates to methods of making such plants and genetically altered plants that display an increased yield.

### FIELD OF THE INVENTION

The invention relates to a method of increasing yield in plants comprising increasing the expression of a nucleic acid encoding a phosphate transporter (PT7) polypeptide. The invention also relates to methods of making such plants and genetically altered plants that display an increased yield.

### BACKGROUND OF THE INVENTION

It is a very urgent issue to sustainably increase crop yields with lower environmental costs to feed the growing population in the world. However, current increase of yields largely depends on extensive application of fertilizers, particularly nitrogen (N) and phosphorus (P) fertilizers^{1,2}. On the other hand, legume plants provide an essential N source through biological N₂ fixation (BNF), and thereby act as a central player in agro-ecosystems³. The huge energy costs imparted during the process of N₂ fixation, and the fast development of nodules both require a sufficient P supply for legumes⁴. Studies have been reported that phosphate (Pi) starvation severely inhibits both nodulation and BNF⁵, with decreased soybean nodule number, nodule size and nitrogenase activity^{6,7}. Therefore, transport of Pi into nodules is a critical process for efficient BNF and nodule organogenesis in legumes.

The process of P transport and/or translocation within plants relies upon various Pi transporters (Pht)⁸. Plant Pi transporters have been classified into five families; Pht1, Pht2, Pht3, Pht4 and pPT⁹⁻¹³. Among these Pi transporters, the members of the Pht1 family are widely studied and well characterized. A number of low- and high-affinity Pht1 family members have been isolated from several plant species, including Arabidopsis¹⁴, rice¹⁵, maize¹⁶ and soybean¹⁷. Generally, low-affinity Pi transporters take part in Pi translocation within organs¹⁸, while high-affinity Pi transporters are mainly involved in Pi uptake from the rhizosphere, and are expressed most strongly in the epidermis and stele of Pi starved roots¹⁹, as well as in cortical cells after mycorrhizal colonization²⁰. However, fewer Pi transporters have been reported to be involved in Pi transport and/or translocation in the legume-rhizobia symbiosis system.

There are two Pi entry pathways in nodules, including a direct pathway from the rhizosphere, and an indirect pathway from host roots to nodules²¹. A previous report has characterized a high-affinity Pi transporter, GmPT5, which controls Pi transport from host roots to nodules in soybean⁶. Meanwhile, mechanisms allowing nodules to directly acquire Pi from the rhizosphere are yet to be uncovered. Furthermore, once Pi is transported and/or taken up into nodules, some of it needs to be translocated into bacteroids for BNF and bacterial requirements. As part of this symbiosis, bacteroids in infected cells of nodules, are surrounded by the plant-derived symbiosome membrane (SM), which is the nutrient exchange interface between the symbionts²². The SM transport proteins in soybean²³, Medicago truncatula²⁴, Lotus japonicas^{25, 26} and other legumes²⁷ have been studied using a range of biochemical and molecular approaches. Transport of calcium has been demonstrated in isolated symbiosomes²⁸, and genes encoding transporters for the movement of iron (GmDMT1)²⁹, nitrate (N70)³⁰, ammonium (GmAMF3)³¹, sulfate (SST1)³², and zinc (GmZIP1)³³ across the SM have also been identified. Recently, the work detailing the SM proteome in soybean has provided a valuable resource for the identification of transporter protein candidates²³. Nevertheless, no Pi transporters acting in the SM have yet been functionally characterized.

There therefore exists a need to increase the yield, particularly the grain yield of legume plants. There also exists a need to increase the nitrogen content of these plants, thereby increasing their value as a source of green manure in agro-ecosystems. The present invention addresses both of these needs.

### SUMMARY OF THE INVENTION

The inventors have identified a dual affinity phosphate (Pi) transporter, PT7 (specifically GmPT7) that is highly expressed in plant root nodules. Interestingly, the inventors have found that this protein is expressed in both the membrane of symbiosomes and the cortical cells of the nodule cortex, and consequently, that overexpression of GmPT7 significantly increases both Pi uptake from the rhizosphere and translocation of Pi across the symbiosome membrane into bacteroids. The inventors have further shown that overexpression of GmPT7 increases nodulation (specifically nodule numbers, size and nitrogenase activity) and more importantly, plant yield. The inventors' findings therefore demonstrate the importance of this transporter in biological nitrogen fixation and show that modulation of this transporter can be used to positively influence plant yield.

In one aspect of the invention, there is provided a method of increasing yield in a plant the method comprising increasing the expression of a nucleic acid sequence encoding a phosphate transporter (PT7) polypeptide.

In one embodiment, the expression of PT7 is increased in at least one root nodule.

In one embodiment, the increase in yield is an increase in seed yield, preferably an increase in seed number. Preferably, the increase in yield is relative to a control or wild-type plant.

In another aspect of the invention there is provided a method of increasing at least one of nodulation, nitrogenase activity, the rate of biological nitrogen fixation, nitrogen content and phosphorous content in a plant, the method comprising increasing the expression of a nucleic acid sequence encoding a phosphate transporter (PT7) polypeptide.

In one embodiment, an increase in nodulation comprises an increase in at least one of nodule number and nodule size.

In one embodiment, said method comprises introducing and expressing in said plant a nucleic acid construct comprising a nucleic acid as defined in SEQ ID NO: 1 or 2 or a homolog or functional variant thereof. Preferably, said nucleic acid is operably linked to a regulatory sequence, and wherein the regulatory sequence is selected from a constitutively active promoter and a nodule-specific promoter.

In a further embodiment, said nucleic acid construct further comprises a nucleic acid sequence encoding a PT5 polypeptide.

In an alternative embodiment, the method comprises introducing a mutation into the plant genome, wherein said mutation is the insertion of at least one or more additional copy of a nucleic acid encoding a PT7 polypeptide or a homolog or variant thereof such that said sequence is operably linked to a regulatory sequence, and wherein such mutation is introduced using targeted genome editing. Preferably, the mutation is introduced using ZFNs, TALENs or CRISPR/Cas9.

In one embodiment, the nucleic acid encoding a PT7 polypeptide comprises or consists of SEQ ID NO or 1 or 2 or a functional variant or homolog thereof. Preferably, said homolog or variant has at least 80% sequence identity to the sequence represented by SEQ ID NO: 1 or 2.

Preferably, the expression of a nucleic acid encoding a PT7 polypeptide is increased relative to a control or wild-type plant.

In another aspect of the invention, there is provided a plant wherein the expression of a nucleic acid encoding a PT7 polypeptide is increased in at least one root nodule compared to the level of expression in a control or wild-type plant. Preferably, said plant expresses a nucleic acid construct comprising a nucleic acid as defined in SEQ ID NO: 1 or 2 or a homolog or functional variant thereof, wherein preferably said construct is operably linked to a regulatory sequence.

In one embodiment, the plant carries a mutation in its genome wherein said mutation is the insertion of at least one or more additional copy of a nucleic acid encoding a PT7 polypeptide or a homolog or a variant thereof such that said sequence is operably linked to a regulatory sequence.

In one embodiment, the regulatory sequence is selected from a constitutively active promoter, a nodule-specific promoter and the endogenous PT7 promoter.

In one embodiment, said mutation is introduced using targeted genome engineering. Preferably, said mutation is introduced using ZFNs, TALENs or CRISPR/Cas9.

In one embodiment, said nucleic acid encoding a PT7 polypeptide consists of SEQ ID NO: 1 or 2 or a functional variant or homolog thereof.

In another aspect of the invention there is provided a method of making a transgenic plant having increased yield, the method comprising introducing and expressing, a nucleic acid construct comprising a nucleic acid as defined in SEQ ID NO: 1 or 2 or a homolog or functional variant thereof in a plant or plant cell.

In one embodiment, the method comprises introducing and expressing the nucleic acid construct in at least one root nodule. Preferably, the nucleic acid further comprises a regulatory sequence, and wherein preferably the regulatory sequence is selected from a constitutively active promoter and a nodule-specific promoter.

In an alternative aspect of the invention there is provided a method of making a genetically altered plant that has increased yield, the method comprising introducing a mutation into the plant genome to increase the expression of a nucleic acid sequence encoding a PT7 polypeptide in at least one root nodule, wherein said mutation is the insertion of at least one or more additional copy of a nucleic acid encoding a PT7 polypeptide or a homolog or variant thereof such that said sequence is operably linked to a promoter, and wherein such mutation is introduced using targeted genome editing. Preferably, the mutation is introduced using ZFNs, TALENs or CRISPR/Cas9.

In one embodiment, the plant is a legume. Preferably, the legume is soybean.

In another aspect of the invention there is provided a plant obtained or obtainable by the method described herein. There is also provided a seed derived from a plant as described herein.

In a further aspect of the invention there is provided the use of a nucleic acid sequence comprising a nucleic acid as defined in SEQ ID NO: 1 or 2 or a homolog or variant thereof to increase yield in a plant.

In another aspect of the invention, there is provided a nucleic acid construct comprising a PT7 nucleic acid sequence and a regulatory sequence, wherein the regulatory sequence is a ENOD40 promoter. Preferably, the PT7 nucleic acid sequence encodes a PT7 polypeptide as defined in SEQ ID NO: 3 or a functional variant or homolog thereof, and wherein the ENOD40 nucleic acid sequence comprises SEQ ID NO: 8 or a functional variant thereof.

There is also provided a vector comprising the nucleic acid sequence described herein and a host cell comprising the nucleic acid construct or the vector described herein.

In another aspect of the invention, there is provided a method of increasing phosphate uptake from the rhizosphere and/or increasing phosphate translocation across the symbiosome membrane, the method comprising increasing the expression of a nucleic acid sequence encoding a phosphate transporter (PT7) polypeptide. Preferably, said nucleic acid encoding a PT7 polypeptide consists of SEQ ID NO: 1 or 2 or a functional variant or homolog thereof.

In another aspect of the invention, there is provided a method for identifying and/or selecting a plant that will have an increase in at least one of yield, nodulation, nitrogenase activity, the rate of biological nitrogen fixation, nitrogen content and phosphorous content, the method comprising screening a population of plants and identifying and/or selecting a plant that has a higher level of PT7 expression than a control plant or a plant from the same or different plant population

In further aspect of the invention, there is provided of the plant as described herein or any part thereof as green manure.

In a final aspect of the invention, there is provided a method of increasing the nitrogen content of a field, the method comprising
(a) growing at least one plant as described herein in the field;
(b) uprooting the plant or part thereof; and
(c) re-ploughing the plant or part thereof into the field.

### DESCRIPTION OF THE FIGURES

The invention is further described in the following non-limiting figures:

FIG. 1 shows immunostaining of GmPT7 protein (red) in nodules of wild-type (WT) nodule (a, j), GmPT7 knockdown (Ri) nodule (b, k) and GmPT7 overexpressing (OX) nodule (c, l). d, e, show magnified images of the yellow box in a; f, g, show magnified images of the yellow box in b; h, i, show magnified images of the yellow box in c; m, n, show magnified images of the yellow box in j; o, p, show magnified images of the yellow box in k; q, r, show magnified images of the yellow box in l. Soybean transgenic plants were grown in low P (LP, a-c), sufficient P (HP, j-l). Blue shows cell wall and nucleus stained by DAPI (yellow arrowheads). CO, cortex; FZ, nitrogen fixation zone. Scale bars, 200 μm.

FIG. 2 shows in vitro assays for radioactive [³³P] Pi uptake and translocation in transgenic nodules. a, [³³P] Pi in the whole nodule. b, [³³P] Pi in symbiosomes. CK, empty vector nodules, OX, GmPT7 overexpressing nodules, Ri, GmPT7 knockdown nodules; LP, low P; HP, sufficient P. Data represent the mean±s.e (n=3). Asterisks indicate significant differences between CK and transgenic lines (Student's t-test, P<0.05). ns, Not significant at 0.05 level. (c)³³Pi uptake in nodules. (d)³³Pi translocation in bacteroids. Soybean transgenic composite plants were pregrown in low P (5 μM KH2PO4) nutrient solution for 50 days, and then nodules were harvested and transferred into 1 mL ³³P labeled nutrient solution containing 0.25 μCi of H3³³PO4 for 2 hours. Ev, empty vector nodules; Ri, GmPT7 knockdown nodules. The corresponding transcripts of GmPT7 in Ri nodules were examined by quantitative real-time (qRT)-PCR. CPM represented radioactive counts per minute measured by a liquid scintillation analyzer. Data are means±SE of three biological replicates from independently transgenic composite lines, and each line contained 20-30 independent transgenic nodules. **Significant at P<0.01, ***Significant at P<0.01 (Student's t-test).

FIG. 3 shows the effect of overexpression or knockdown of GmPT7 on soybean nodulation. a, Nodule growth performance; b, nodule number; c, nodule fresh weight; d, nitrogenase activity of different lines. WT, wild-type, OX, over-expressing lines, Ri, knockdown lines, LP, low P, HP, sufficient P. Rep, replication. Scale bars, 3 cm. Data represent the mean±s.e (n=3). Asterisks indicate significant differences between WT and transgenic lines (Student's t-test, P<0.05). ns, Not significant at 0.05 level. The whole experiment had been independently repeated twice, and the results showed the similar tendency.

FIG. 4 shows GmPT7 expression level affects development of transgenic soybean nodules. (a, b) Fixation zone in section of nodules were stained by toluidine blue. Bar, 100 μm. (c, d) Surface area of 100 infected cells. Soybean whole transgenic plants were grown in hydroponics under low P (LP, 5 μM KH₂PO₄) and sufficient P conditions (HP, 250 μM KH₂PO₄). Data represent the mean±SE from ten independently biological replications. ***Significant at P<0.01 (Student's t-test).

FIG. 5 shows the effects of overexpression or knockdown of GmPT7 on soybean yield in the field. a, Soybean growth performance. b, Seed number. c, Yield. WT, wild-type, OX, over-expressing lines, Ri, knockdown lines. Scale bars, 20 cm. Data represent the mean±s.e (n=30). Asterisks indicate significant differences between WT and transgenic lines (Student's t-test, P<0.05). ns, Not significant at 0.05 level.

FIG. 6 shows the effects of double suppression of GmPT5 and GmPT7 on nodulation of transgenic composite soybeans under low P conditions. a-c, Nodules growth performance. c, Bacteriod carried GFP in infected cells in transgenic nodules. d, Nodule number; e, nodule fresh weight; f, plant fresh weight; g, plant nitrogen content of different lines. CK, empty vector, Ri, GmPT5 and GmPT7 double suppressed lines. Rep, replication. Scale bars, a, b, 2 cm, c, 20 μm. Data represent the mean±s.e (n=3). Asterisks indicate significant differences between CK and Ri lines (Student's t-test, P<0.05).

FIG. 7 shows a, the expression patterns of GmPT genes in soybean nodules under sufficient P conditions. b, Expression patterns of GmPT7 in different soybean tissues. LP, low P, HP, sufficient P. Data represent the mean±s.e (n=4).

FIG. 8 Phosphate transport activity of soybean GmPT7 in yeast mutants. (a) Staining of the yeast MB192 mutant transformed with GmPT7 by Bromocresol purple. (b) Rate of 33P transport by MB192-GmPT7 at different Pi concentrations. MB192, the yeast mutant defective in Pi uptake, which harboring an empty vector Yp112 as negative control; MB192 (GmPT7), GmPT7 fused with vector Yp112 in MB192; MB192 (PHO84), PHO84 (a Pi transporter) fused with vector Yp112 in MB192 as positive control. The GmPT7 mediated 33Pi uptake velocities after subtracting the Pi transport with an empty vector following the Michaelis-Menten kinetics equation.

FIG. 9 shows the subcellular localization of GmPT7. a-h, Localization of the GFP-GmPT7 fusion protein (a-d), and GFP (e-h), transiently expressed in protoplasts prepared from Arabidopsis leaves. a,e, GFP image; b,f, chlorophyll fluorescence; c,g, bright field image; and d,h, a combined image of the three channels. Scale bars, 40 μm. i, Subcellular localization of GmPT7 fused to GFP in epidermal onion cells. Plasmolysis in epidermal onion cells was induced by adding 30% sucrose solution prior to confocal observation. GFP, PI, OL and BF in the images stand for green fluorescence, red propidium iodide (PI) fluorescence, overlaid by former three pictures and bright field. Scale bars, 100 μm.

FIG. 10 shows the tissue- and cell-specificity of GmPT7 localization in nodules under sufficient P conditions. a, GUS staining in proGmPT7::GUS transgenic soybean nodules. b, c, Immunostaining of GmPT7 protein (red) in soybean nodules. c, The magnified image of the yellow box in b. CO, cortex, FZ, nitrogen fixation zone. Scale bars, 200 μm.

FIG. 11 shows the relative expression of GmPT7 in soybean whole transgenic plants. Plants were grown in hydroponics under low P (a) (LP, 5 μM KH2PO4) and sufficient P conditions (b) (HP, 250 μM KH2PO4). WT, wild-type; OX, GmPT7 overexpressing lines; Ri, GmPT7 knockdown lines. The total RNA was extracted from nodules. Data represent the mean±SE from three independently biological replications. *Significant at P<0.05, **Significant at P<0.01, ***Significant at P<0.01 (Student's t-test). (c) Relative expression of GmPT5 and GmPT7 in soybean transgenic composite plants. Ev, empty vector; DRi, GmPT5 and GmPT7 double suppressed lines. Plants were inoculated with rhizobia, and then transplanted into nutrient solution with 5 μM P and 500 μM N supply for 30 days. Data are means±SE of three biological replicates from independently transgenic composite lines. * Significant at P<0.05, ** Significant at P<0.01 (Student's t-test).

FIG. 12 shows the effects of overexpression (OX) or knockdown (Ri) of GmPT7 on soybean growth. a, plant fresh weight; b, plant nitrogen content; c, plant phosphorous content; d, relative expression of GmPT7 of different lines. WT, wild-type. LP, low P, HP, sufficient P. Data represent the mean±s.e (n=3). Asterisks indicate significant differences between WT and transgenic lines (Student's t-test, P<0.05). ns, Not significant at 0.05 level.

FIG. 13 shows a proposed model of Pi uptake and translocation cooperatively controlled by two Pi transporters, GmPT5 and GmPT7, in soybean nodules. Pi entry into nodules through two pathways, including an indirect pathway \textcircled{1} and a direct pathway \textcircled{2}. For the indirect pathway, Pi is transported from host roots to nodules via vascular tissues and is controlled by GmPT5. For the direct pathway, Pi is directly absorbed from the rhizosphere into nodules via GmPT7. GmPT7 is also responsible for Pi translocation between symbionts across the SM of infected cells \textcircled{3}.

FIG. 14 shows the effects of overexpression or knockdown of GmPT7 on soybean pod number and grain weight in the field. WT, wild-type, OX, over-expressing lines, Ri, knockdown lines. Data represent the mean±s.e (n=30). Asterisks indicate significant differences between WT and transgenic lines (Student's t-test, P<0.05). ns, Not significant at 0.05 level.

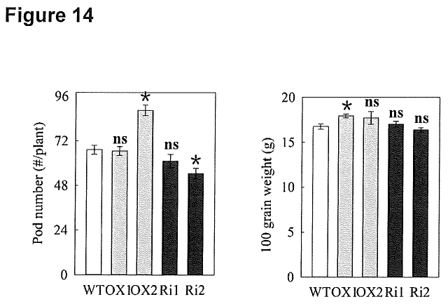

FIG. 15 shows that overexpression of GmPT7 increased soybean yield by up to 36%. Plants inoculated with rhizobia were sown on acidic soils, and pods were harvested for yield evaluation at maturation stage. WT, wild-type; OX, GmPT7 overexpressing lines; Ri, GmPT7 knockdown lines. Data represent the mean±SE from thirty transgenic plants. *Significant at P<0.05, **Significant at P<0.01, ***Significant at P<0.01 (Student's t-test).

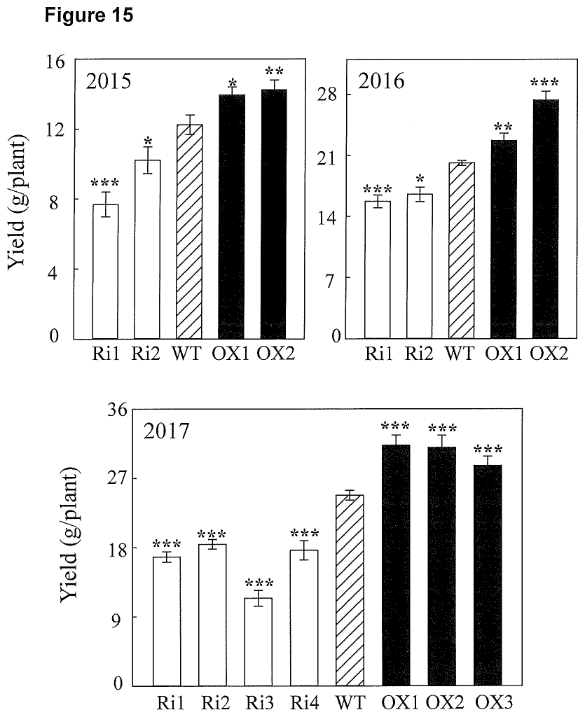

FIG. 16 shows a correlation between GmPT7 expression in nodules and nodule number, pod number and seed weight of soybean in the field. Two populations are shown; a core collection with 194 germplasms and a population of Recombinant Inbred Lines (RILs) with 103 progenies. Except the correlation with nodule number in RILs, all the correlations are significant. (a) correlation between GmPT7 expression and nodule number in the core collection. Correlation coefficient is 0.305, P value is 0.000130 and the number of samples was 153. (b) correlation between GmPT7 expression and pod number in the core collection. Correlation coefficient is 0.303, P value is 0.000131 and the number of samples was 154. (c) correlation between GmPT7 expression and soybean yield in the core collection. Correlation coefficient is 0.308, P value is 0.0000566 and the number of samples was 165. (d) correlation between GmPT7 expression and nodule number in the RILs. Correlation coefficient is 0.135, P value is 0.228 and the number of samples was 81. (e) correlation between GmPT7 expression and pod number in the RILs. Correlation coefficient is 0.322, P value is 0.00381 and the number of samples was 79. (f) correlation between GmPT7 expression and soybean yield in the RILs. Correlation coefficient is 0.313, P value is 0.00582 and the number of samples was 76.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry, recombinant DNA technology and bioinformatics which are within the skill of the art. Such techniques are explained fully in the literature.

The terms “seed”, “grain” and “bean” as used herein can be used interchangeably.

As used herein, the words “nucleic acid”, “nucleic acid sequence”, “nucleotide”, “nucleic acid molecule” or “polynucleotide” can be used interchangeably and are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place.

The aspects of the invention involve recombination DNA technology and exclude embodiments that are solely based on generating plants by traditional breeding methods.

As used herein, the terms “increasing the expression” means an increase in the nucleotide and/or protein levels of PT7.

For the purposes of the invention, a “mutant” plant is a plant that has been genetically altered compared to the naturally occurring wild type (WT) plant. In one embodiment, a mutant plant is a plant that has been altered compared to the naturally occurring wild type (WT) plant using a mutagenesis method, such as the mutagenesis methods described herein. In one embodiment, the mutagenesis method is targeted genome modification or genome editing. In one embodiment, the plant genome has been altered compared to wild type sequences using a mutagenesis method. In one example, mutations can be used to insert a PT7 gene sequence to enhance levels of expression of a PT7 nucleic acid compared to a wild-type plant. In one example, the PT7 sequence is operably linked to an endogenous promoter. Such plants have an altered phenotype as described herein, such as an increased seed yield. Therefore, in this example, increased seed yield is conferred by the presence of an altered plant genome and is not conferred by the presence of transgenes expressed in the plant.

In one aspect of the invention, there is provided a method for increasing yield in a plant, the method comprising increasing the expression of a nucleic acid sequence that encodes a phosphate transporter (PT7) polypeptide.

The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

Thus, according to the invention, yield comprises one or more of and can be measured by assessing one or more of: increased seed yield per plant, increased seed filling rate, increased number of filled seeds, increased harvest index, increased viability/germination efficiency, increased number or size or weight of seeds or pods or beans or grain, increased growth or increased branching, for example inflorescences with more branches, increased biomass, increased fresh weight or grain fill. Preferably, increased yield comprises at least one of an increased number or weight of seeds, beans or pods per plant, increased thousand kernel weight (TKW), increased biomass, increased fresh weight and increased growth. Yield is increased relative to a control or wild-type plant. For example, the yield is increased by 2%, 3%, 4%, 5%-50% or more compared to a control plant, for example by at least 10%, 15%, 20%, 25%, 30%, 35%, 40%, 45% or 50%.

In one embodiment, the method comprises increasing the expression of a nucleic acid sequence encoding a PT7 polypeptide in at least or solely in the root nodules of the plant. In a further preferred embodiment, the expression of PT7 is increased in at least one root nodule, and in at least one, preferably both, of the cortical cells of the root nodule and the symbiosome membrane. Accordingly, in one embodiment, the method comprises increasing the expression of PT7 in at least one root nodule, and within the root nodule, more preferably in at least one, preferably both of the cortical cells and the symbiosome membrane. In one embodiment, the expression of PT7 is increased only in the root nodule, preferably the cortical cells and/or the symbiosome membrane. Accordingly, in one embodiment, the method may further comprise the step of measuring the level of PT7 expression in at least one root nodule, and preferably comparing said level to the level of expression in a wild-type or control plant. Techniques to measure the level of PT7 in root nodules are well known to the skilled person.

In a further embodiment or aspect of the invention there is provided a method of increasing at least one of nodulation, nitrogenase activity, the rate of biological nitrogen fixation (BNF), total nitrogen content and phosphorus content of the plant. In one embodiment, said increase is at least 10%, 15%, 20%, 25%, 30%, 35%, 40%, 45%, 50%, 60%, 70%, 80%, 90%, 100%, 110%, 120% or 130% compared to a control or wild-type plant. In one embodiment, the method comprises increasing at least one of nodulation, nitrogenase activity, the rate of biological nitrogen fixation (BNF), total nitrogen content and phosphorus content in addition to increasing yield in a plant.

By “nodulation” is meant nodule development, and an increase in nodulation can be reflected in an increase in the number and/or weight of nodules per plant. In one embodiment, nodulation is increased by at least 30%, 40%, 50%, 55%, 60%, 65% or 70% compared to a control or wild-type plant.

By “nitrogenase activity” is meant the activity of the Rhizobia nitrogenase enzyme, which converts nitrogen into ammonia and H₂. Methods of measuring nitrogenase activity would be well known to the skilled person. For example, the rate at which the end-product, H₂, is produced by nodules can be used as a means to measure nitrogenase activity. Alternatively, the rate at which nitrogenase can reduce acetylene into ethylene can be used as a measure of nitrogenase activity (the “acetylene reduction method”, as described in David et al. 1980 is incorporated herein by reference 34). By “biological nitrogen fixation” or BNF is meant the rate at which nitrogen is converted to ammonia and incorporated into plant tissue. In one embodiment, nitrogenase activity is increased by at least 20%, 30%, 40%, 50%, 60%, 70%, 80%, 90%, 100%, 110%, 120% or 130% compared to a control or wild-type plant.

In another aspect of the invention, there is provided a method of increasing the uptake of phosphate into roots from the rhizosphere and/or increasing phosphate translocation across the symbiosome membrane, wherein the method comprises increasing the expression of a nucleic acid sequence encoding a PT7 polypeptide. Preferably, said method comprises increasing the uptake of phosphate from the rhizosphere and increasing the translocation of phosphate across the symbiosome membrane. In this embodiment, an increase is the uptake of phosphate from the rhizosphere results in an increase in phosphate uptake into nodules. Similarly, an increase in phosphate translocation can be measured by measuring total phosphate uptake into the symbiosome. In one embodiment, said increase is at least 10%, 15%, 20%, 25%, 30%, 35%, 40%, 45%, 50%, 60%, 70%, 80%, 90%, 100%, 110%, 120% or 130% compared to a control or wild-type plant.

The terms “increase”, “improve” or “enhance” according to the various aspects of the invention can be used interchangeably.

In one embodiment, the above described phenotypes are observed irrespective of whether the plant is grown under phosphate sufficient or deficient conditions. In general, an exchangeable soil P concentration lower than 10 mg/kg (ppm) could be considered as P deficient for most plants. In this case, over 40% of arable land soil would be P deficient, especially in tropic and subtropical areas (Kochian et al, 2004).

In one embodiment, the method comprises introducing and expressing in the plant a nucleic acid construct comprising a PT7 nucleic acid. In one embodiment, the PT7 nucleic acid sequence encodes a PT7 polypeptide as defined in SEQ ID NO: 3. In a further preferred embodiment, the PT7 nucleic acid sequence comprises or consists of SEQ ID NO: 1 or 2 or a homologue or variant thereof. In one embodiment, PT7 is soybean PT7, or GmPT7.

In one embodiment, the nucleic acid sequence may be expressed using a promoter that drives overexpression. Overexpression according to the invention means that the transgene is expressed at a level that is higher than the expression of the endogenous PT7 gene whose expression is driven by its endogenous counterpart. In one embodiment, overexpression may be driven by a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression.

In an alternative embodiment, the regulatory sequence is a tissue specific promoter. Tissue specific promoters are transcriptional control elements that are only active in particular cells or tissues at specific times during plant development. In a preferred embodiment, the promoter is a nodule-specific promoter. For example, the promoter may be the endogenous PT7 promoter or GmPT7 (SEQ ID NO: 4). Alternatively, the promoter may be a nodule-specific promoter such as endogenous ENOD40 (early nodulin 40) promoter. Accordingly, in one embodiment, the nodule-specific promoter is ENOD40, which comprises or consists of a sequence as defined in SEQ ID NO: 8 or a functional variant thereof. A functional variant is as defined herein.

In one embodiment, the nucleic acid and regulatory sequence are from the same plant family. In another embodiment, the nucleic acid and regulatory sequence are from a different plant family, genus or species.

According to all aspects of the invention, including the method above and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

A “plant promoter” comprises regulatory elements which mediate the expression of a coding sequence segment in plant cells. The promoters upstream of the nucleotide sequences useful in the nucleic acid constructs described herein can also be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoter is increased by modification of their sequence, or that they are replaced completely by more active promoters, even promoters from heterologous organisms. For expression in plants, the PT7 nucleic acid sequence is, as described above, preferably linked operably to or comprises a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

The term “operably linked” as used herein refers to a functional linkage between the promoter sequence and the gene of interest, such that the promoter sequence is able to initiate transcription of the gene of interest.

In a further embodiment, the nucleic acid construct may further comprise a nucleic acid sequence encoding a second phosphate transporter. In one embodiment, the second phosphate transporter is PT5, more preferably soybean PT5 or GmPT5. Preferably, the nucleic acid sequence of PT5 comprises or consists of SEQ ID NO: 5 or 6 and encodes a polypeptide as defined in SEQ ID NO: 7. Alternatively, the method may comprise introducing and expressing a second nucleic acid construct comprising a second phosphate transporter, wherein preferably the second phosphate transporter is PT5 as defined herein. In this embodiment, the second nucleic acid construct may be introduced and expressed in the plant before, after or concurrently with the first nucleic acid construct.

In one embodiment, the progeny plant is stably transformed with the nucleic acid construct described herein and comprises the exogenous polynucleotide which is heritably maintained in the plant cell. The method may include steps to verify that the construct is stably integrated. The method may also comprise the additional step of collecting seeds from the selected progeny plant.

In an alternative embodiment of the invention, the method comprises introducing a mutation into the plant genome, wherein said mutation is the insertion of at least one or more additional copy of a nucleic acid encoding a PT7 polypeptide or a homolog or variant thereof and as defined herein such that said sequence is operably linked to a regulatory sequence, and wherein such mutation is introduced using targeted genome editing. In one embodiment the regulatory sequence is the endogenous PT7 promoter. Preferably said mutation results in an increase in the expression of the PT7 nucleic acid relative to a control or wild-type plant. Preferably said mutation results in an increase in the expression of PT7 in at least one root nodule and more preferably in at least one of or both of the cortical cells of the nodule cortex and the symbiosome membrane within the root nodule.

In a further embodiment, the method may further comprise introducing a second mutation into the plant genome, wherein the mutation is the insertion of at least one or more additional copy of a nucleic acid encoding a PT5 polypeptide or a homolog or variant thereof and as defined herein such that said sequence is operably linked to a regulatory sequence, and wherein such mutation is introduced using targeted genome editing.

In one embodiment, the mutation is introduced using ZFNs, TALENs or CRISPR/Cas9.

In a further embodiment, the method may further comprise at least one or more of the steps of assessing the phenotype of the transgenic or genetically altered plant, measuring at least one of yield traits, preferably seed number, biomass, fresh weight, nodulation, the rate of biological nitrogen fixation, phosphorus content and/or nitrogen content, and comparing said phenotype to determine an increase in at least one of yield, preferably seed number, biomass, fresh weight, nodulation, the rate of biological nitrogen fixation, phosphorus content and/or nitrogen content in a wild-type or control plant. In other words, the method may involve the step of screening the plants for the desired phenotype. In a further embodiment, the method may further comprise screening the plants for an increased level of PT7 expression, wherein said increase is relative to a control or wild-type plant.

In one embodiment, the expression of a nucleic acid encoding a PT7 polypeptide is increased relative to a control or wild-type plant. Preferably said increase is at least 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold or at least 150-fold higher than the level of expression in a control or wild-type plant. As already discussed, techniques for measuring the desired nucleic acid or protein expression levels are well known in the art.

The invention also relates to a plant, preferably a transgenic or mutant or genetically altered plant, characterised in that the expression of PT7 is increased compared to the level of expression in a control or wild-type plant.

In one embodiment, the expression of PT7 is increased in at least one root nodule compared to the level of expression in a control or wild-type plant. Preferably, said expression is increased in all root nodules. In particular, within the at least one root nodule, expression of PT7 is increased in at least one, preferably both of the cortical cells of the nodule cortex and the symbiosome membrane. In a specific embodiment, the expression of PT7 may be increased in at least 10%, 20%, 30%, 40%, 50%, 60%, 70%, 80%, 90%, 95% or 100% of root nodules.

In a further embodiment, said increase is at least 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold or at least 150-fold higher than the level of expression in a control or wild-type plant. As already discussed, techniques for measuring the desired nucleic acid or protein expression levels are well known in the art.

The plant is also characterised in that it shows an increase in at least one of yield, seed number, biomass, fresh weight nodulation, rate of biological nitrogen fixation, nodule number, nodule size, nitrogenase activity, phosphorus content and nitrogen content. Such increase is at least 10%, 15%, 20%, 25%, 30%, 35%, 40%, 45%, 50%, 60%, 70%, 80%, 90%, 100%, 110%, 120% or 130% compared to a control or wild-type plant.

In one embodiment, the plant expresses a polynucleotide “exogenous” to said plant, that is a polynucleotide which is introduced into the plant by any means other than by a sexual cross. Examples of means by which this can be accomplished are described below. In one embodiment of the method, an exogenous nucleic acid is expressed in the transgenic plant which is a nucleic acid construct comprising a nucleic acid as defined in SEQ ID NO: 1 or 2 or a homolog or functional variant thereof that is not endogenous to said plant but is from another plant species. For example, the GmPT7 construct can be expressed in another plant or legume that is not soybean.

In an alternative embodiment, an endogenous nucleic acid construct is expressed in the transgenic plant. For example, the GmPT7 construct can be expressed in soybean. Accordingly, in one embodiment, the plant expresses a nucleic acid comprising a nucleic acid as defined in SEQ ID NO: 1 or 2 or a homolog or functional variant thereof.

In another embodiment, the plant expresses an exogenous or endogenous nucleic acid sequence encoding a second phosphate transporter. In one embodiment, the second phosphate transporter is PT5 more preferably soybean PT5 or GmPT5. Preferably, the nucleic acid sequence of PT5 comprises or consists of SEQ ID NO: 5 or 6 and encodes a polypeptide as defined in SEQ ID NO: 7.

In an alternative embodiment, the plant carries a mutation in its genome, wherein said mutation is the insertion of at least one or more additional copy of a nucleic acid encoding a PT7 polypeptide or a homolog or a variant thereof such that said sequence is operably linked to a regulatory sequence. Preferably, said mutation is introduced using targeted genome modification and more preferably, said mutation is introduced using ZFNs, TALENs or CRISPR/Cas9. In a further embodiment, the plant may further comprise a second mutation in the plant genome, wherein the mutation is the insertion of at least one or more additional copy of a nucleic acid encoding a PT5 polypeptide or a homolog or variant thereof and as defined herein such that said sequence is operably linked to a regulatory sequence, and wherein such mutation is introduced using targeted genome editing.

Preferably, the nucleic acid sequence is operably linked to a regulatory sequence. A regulatory sequence may be as defined above.

In another aspect of the invention, there is provided a method of making a transgenic plant, characterised in that the plant shows an increase in yield, the method comprising introducing and expressing a nucleic acid construct comprising a nucleic acid as defined in SEQ ID NO: 1 or 2 or a homolog or functional variant thereof in a plant or plant cell.

In a further embodiment, the nucleic acid construct may further comprise a nucleic acid sequence encoding a second phosphate transporter. In one embodiment, the second phosphate transporter is PT5 more preferably soybean PT5 or GmPT5. Preferably, the nucleic acid sequence of PT5 comprises or consists of SEQ ID NO: 5 or 6 and encodes a polypeptide as defined in SEQ ID NO: 7. Alternatively, the method may comprise introducing and expressing a second nucleic acid construct comprising a second phosphate transporter, wherein preferably the second phosphate transporter is PT5 as defined herein. In this embodiment, the second nucleic acid construct may be introduced and expressed in the plant before, after or concurrently with the first nucleic acid construct.

The method may further comprise regenerating a transgenic plant from the plant or plant cell wherein the transgenic plant comprises in its genome a nucleic acid sequence selected from SEQ ID NO: 1 or 2 or a nucleic acid that encodes a PT7 protein as defined in SEQ ID NO: 3 and obtaining a progeny plant derived from the transgenic plant, wherein said progeny exhibits at least one of an increased yield, seed number, biomass, fresh weight, nodulation, rate of biological nitrogen fixation, nodule number, nodule size, nitrogenase activity, phosphorus content and nitrogen content.

In a further embodiment, the method may further comprise at least one or more of the steps of assessing the phenotype of the transgenic or genetically altered plant, measuring at least one of yield, preferably seed number, biomass, fresh weight, nodulation, the rate of biological nitrogen fixation, phosphorus content and/or nitrogen content, and comparing said phenotype to determine an increase in at least one of yield, preferably seed number, biomass, fresh weight, nodulation, the rate of biological nitrogen fixation, phosphorus content and/or nitrogen content in a wild-type or control plant. In other words, the method may involve the step of screening the plants for the desired phenotype.

Transformation methods for generating a transgenic plant of the invention are known in the art. Thus, according to the various aspects of the invention, a nucleic acid construct as defined herein is introduced into a plant and expressed as a transgene. The nucleic acid construct is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker. Following DNA transfer and regeneration, putatively transformed plants may also be evaluated, for instance using Southern analysis, for the presence of the gene of interest, copy number and/or genomic organisation. Alternatively or additionally, RNA and protein expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In another aspect of the invention, there is provided a method of producing a mutant or genetically altered plant, the method comprising introducing a mutation into the plant genome, wherein said mutation is the insertion of at least one or more additional copy of a nucleic acid encoding a PT7 polypeptide or a homolog or variant thereof such that said sequence is operably linked to a regulatory sequence, and wherein such mutation is introduced using targeted genome editing. Preferably, the plant is characterised in that the plant shows an increase in one of the desired phenotypes described herein. For example, the plant shows an increase in yield. Alternatively, the plant is characterised in that the plant shows an increase in PT7 expression in at least one root nodule, as described herein, and within the nodule, in at least one, preferably both, of the cortical cells of the nodule cortex and the symbiosome membrane.

In a further embodiment, the method may further comprise introducing a second mutation into the plant genome, wherein the mutation is the insertion of at least one or more additional copy of a nucleic acid encoding a PT5 polypeptide or a homolog or variant thereof and as defined herein such that said sequence is operably linked to a regulatory sequence, and wherein such mutation is introduced using targeted genome editing.

In a preferred embodiment, the mutation is introduced by mutagenesis or targeted genome editing.

The regulatory sequence may be the endogenous PT7 promoter. In the above embodiments an “endogenous” nucleic acid may refer to the native or natural sequence in the plant genome.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Cas9 expression plasmids for use in the methods of the invention can be constructed as described in the art.

Thus, aspects of the invention involve targeted mutagenesis methods, specifically genome editing, and in a preferred embodiment exclude embodiments that are solely based on generating plants by traditional breeding methods.

The invention also extends to a plant obtained or obtainable by any method described herein.

In another embodiment, the nucleic acid encoding a PT7 polypeptide comprises or consists of a sequence as defined in SEQ ID NO 1 or 2 or a functional variant or homolog thereof and encodes a PT7 protein as defined in SEQ ID NO:3 or a functional variant or homolog thereof. In one embodiment, the PT7 is GmPT7 (i.e. Glycine max PT7).

The term “functional variant of a nucleic acid sequence” as used herein with reference to any of SEQ ID Nos 1, 2 or 3 refers to a variant gene sequence or part of the gene sequence which retains the biological function of the full non-variant sequence, for example confers at least one of increased yield, seed number, biomass, fresh weight, nodulation, rate of biological nitrogen fixation, phosphorus content and/or nitrogen content when expressed in a plant. A functional variant also comprises a variant of the gene of interest which has sequence alterations that do not affect function, for example in non-conserved residues. Also encompassed is a variant that is substantially identical, i.e. has only some sequence variations, for example in non-conserved residues, compared to the wild type sequences as shown herein and is biologically active.

Thus, it is understood, as those skilled in the art will appreciate, that the aspects of the invention, including the methods and uses, encompasses not only a nucleic acid sequence or amino acid sequence comprising or consisting a sequence selected from SEQ ID Nos 1-3 but also functional variants or parts of these SEQ ID NOs that do not affect the biological activity and function of the resulting protein. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homologue as used herein also designates an GmPT7 orthologue from another plant species. A homologue of a GmPT7 polypeptide or a GmPT7 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NOs: 1, 2 or 3. In one embodiment, overall sequence identity is at least 37%. In one embodiment, overall sequence identity is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%.

The term “PT7” refers to a plasma membrane-localised phosphate transporter, phosphate transporter 7, which the inventors have surprisingly demonstrated to be a dual-affinity (or wide affinity, such terms may be equivalent in this context) phosphate transporter that is expressed in both the symbiosome membrane and the cortical cells of the nodule cortex.

Functional variants of GmPT7 homologs are also within the scope of the invention.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Thus the GmPT7 nucleotide and/or amino acid sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologues. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In one embodiment, there is provided a method of increasing yield, nodulation, nitrogenase activity, the rate of biological nitrogen fixation, nitrogen content and phosphorous content in a plant, as described herein, the method comprising increasing the expression of PT7, wherein the PT7 gene comprises or consists of
a. a nucleic acid sequence encoding a polypeptide as defined in SEQ ID NO:3; or
b. a nucleic acid sequence as defined in SEQ ID NO: 1 or 2; or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a PT7 polypeptide as defined herein that is capable of hybridising under stringent conditions as defined herein to the nucleic acid sequence of any of (a) to (d).

In another aspect of the invention, there is provided a nucleic acid construct comprising a PT7 nucleic acid and a regulatory sequence. In one embodiment, the PT7 nucleic acid encodes a PT7 polypeptide as defined in SEQ ID NO: 3. In a further embodiment, the PT7 nucleic acid sequence comprises or consists of a nucleic acid sequence as defined in SEQ ID NO: 1 or 2 or a homolog or functional variant thereof. In a preferred embodiment, the regulatory sequence is the ENOD40 promoter. In a further preferred embodiment, the ENOD40 sequence comprises or consists of a nucleic acid sequence as defined in SEQ ID NO: 8. A functional variant or homolog is described above. In one embodiment, the nucleic acid and regulatory sequence are from the same plant family. In another embodiment, the nucleic acid and regulatory sequence are from a different plant family, genus or species. In a further aspect of the invention, there is provided a vector comprising the nucleic acid construct as defined herein.

In another aspect, the invention relates to an isolated host cell transformed with a nucleic acid construct or vector as described above. The host cell may be a bacterial cell, such as Agrobacterium tumefaciens, or an isolated plant cell. The invention also relates to a culture medium or kit comprising a culture medium and an isolated host cell as described herein.

The nucleic acid construct or vector described above can be used to generate transgenic plants using transformation methods known in the art and described herein.

Thus, in a further aspect, the invention relates to a transgenic plant expressing the nucleic acid construct as described herein

In another aspect of the invention there is provided the use of a nucleic acid or nucleic acid construct as described herein to increase yield in a plant. Alternatively, there is provided the use of a nucleic acid or nucleic acid construct to increase at least one of yield, seed number, biomass, nodulation (e.g. nodule number, nodule size,), rate of biological nitrogen fixation, nitrogenase activity, phosphorus content and nitrogen content.

In a further aspect of the invention, there is provided the use of a plant as defined herein as green manure. It has been known for centuries that legumes can increase the yield of other crops when they are grown in rotation. The plants of the invention, that are characterised by an increased nitrogen content (compared to wild-type or control plants), can serve as green manure by leaving the uprooted plant or sown plant parts of the invention to wither on a field to serve as a mulch and/or a soil conditioner. Typically plants used as green manure are ploughed under and incorporated into the soil while green or shortly after flowering.

Therefore, in a related aspect of the invention, there is provided a method of increasing the nitrogen content (i.e. total nitrogen content) of a field (i.e. the soil of a field) the method comprising (a) growing at least 30 plants as defined herein in the field, (b) uprooting the plant or part thereof, preferably while green or after flowering, and (c) re-ploughing the plant or part thereof into the field. In one embodiment, the nitrogen content of the field is increased compared to a field where a plant or part thereof of the present invention has not been grown in the field and re-ploughed as described above.

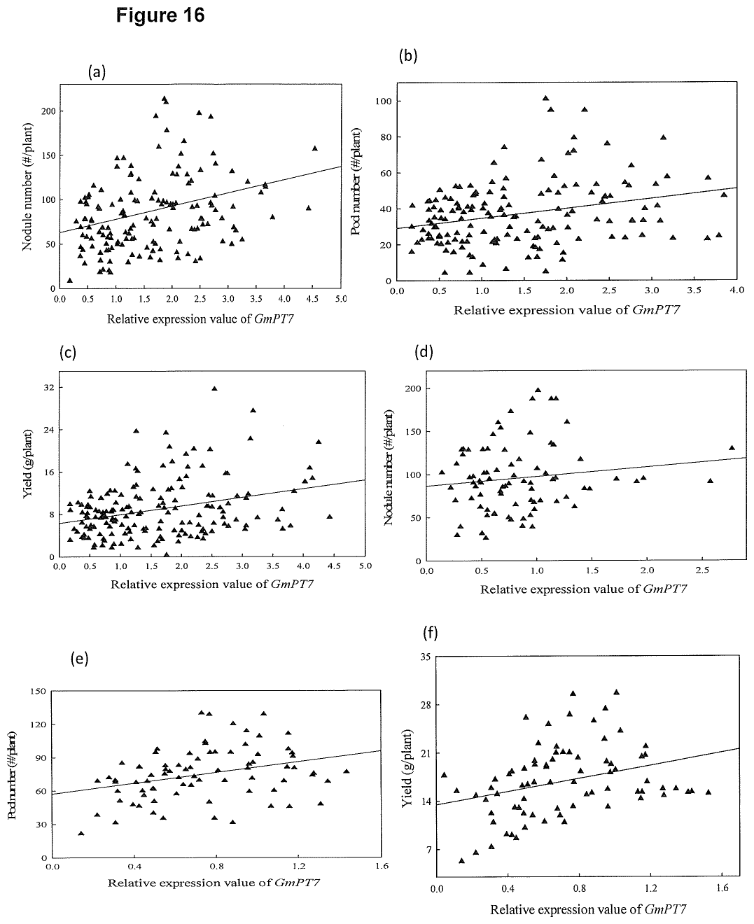

The inventors have further identified that there exists within a population of plants of the same species, a natural variation in the levels of PT7 protein, and moreover, as shown in FIG. 16, that an increase in PT7 expression levels is associated with an increase in nodule number, pod number and yield.

Accordingly, in another aspect of the invention, there is provided a method for screening a population of plants and identifying and/or selecting and/or breeding a plant that has a level of PT7 expression, preferably in its germplasm, that is higher level than the level of PT7 expression in at least one other plant in the same or different plant population. The method may further comprise selecting said plant for further propagation. In one embodiment, RT-PCR may be used to measure expression levels, although other techniques would be known to the skilled person. In another embodiment, the method may comprise comparing the expression level to a control plant. Preferably said increase is at least 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold or at least 150-fold higher than the level of expression in the at least one other plant in the plant population or the control plant. Preferably said selected plant has the highest level of PT7 expression in the plant population. As a result, such plants will display increased yield and/or increased nodulation and/or increased nitrogenase activity and/or increased phosphorous and/or nitrogen content as described herein. The method may further comprise collecting seed from the selected plant.

In a further aspect of the invention there is provided a method for increasing and/or increased nodulation and/or increased nitrogenase activity and/or increased phosphorous and/or nitrogen content, in a plant, the method comprising
a. screening a population of plants for at least one plant with an increased or decreased level of PT7 expression compared to a control plant;
b. further increasing the expression of a PT7 polypeptide, as described herein.

A plant according to the various aspects of the invention, including the transgenic plants, methods and uses described herein may be a dicot plant. Preferably, the plant is a crop plant. By crop plant is meant any plant which is grown on a commercial scale for human or animal consumption or use. In a preferred embodiment, the plant is a cereal. Most preferred plants are legumes, such as but not limited to soybean, pea, peanut and the common bean (Phaseolus vulgaris). The most preferred plant is soybean.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. The invention also relates to food products and food supplements comprising the plant of the invention or parts thereof. In another aspect of the invention, there is provided a product derived from a plant as described herein or from a part thereof. In a preferred embodiment, the harvestable part is the seed, bean or pod.

A control plant as used herein according to all of the aspects of the invention is a plant which has not been modified according to the methods of the invention. Accordingly, in one embodiment, the control plant does not have an altered expression profile of a PT7 nucleic acid. In an alternative embodiment, the control plant does not express the nucleic acid construct described herein, nor has the plant been genetically modified, as described above. In one embodiment, the control plant is a wild type plant. The control plant is typically of the same plant species, preferably having the same genetic background as the modified plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### Example

Soybean [Glycine max (L.) Merr.] is one of the most world widely grown leguminous crops, and has a superior BNF capacity³⁵. In this study, a soybean SM localized Pi transporter, GmPT7 was identified and found to be critically involved in both Pi uptake from the rhizosphere and Pi translocation from host plant cells to bacteroids. Double suppression of GmPT5 and GmPT7 severely inhibited soybean nodulation. By contrast, overexpression of GmPT7 improved the nodule development and nitrogenase activity, and subsequently changes soybean yield in the field. To the best of our knowledge, these results constitute a discovery of a Pi transporter acting in direct Pi uptake into nodules and Pi translocation in symbionts across SM, while also demonstrate that GmPT5 and GmPT7 together control the dominant Pi uptake and translocation in nodules, and thus influence N2 fixation and productivity of soybean.

Identification of a Nodule-Expressed Pi Transporter Gene, GmPT7.

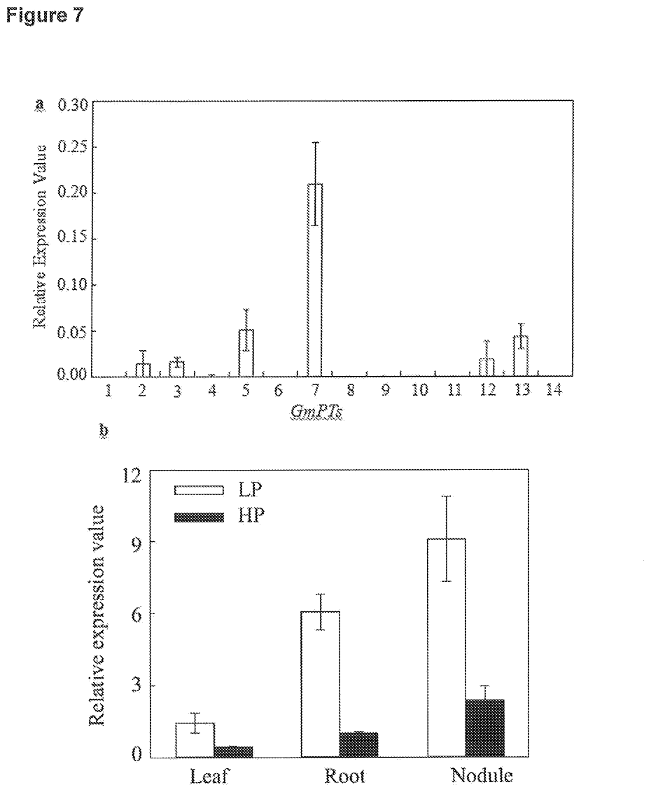

Fourteen members of the Pht1 family (GmPT1-GmPT14) were identified in the soybean genome as previously described¹⁷ Combining GmPTs with Pi transporter protein sequences from algae, Arabidopsis, rice, legume and other species, we constructed a phylogenetic tree using neighbour-joining analysis in the MEGA 5.05 program, and found that 4 GmPTs (GmPT1, GmPT4, GmPT7 and GmPT13) were clustered in a legume-specific subgroup, suggesting that these members might play special roles in legumes. However, none of them has yet been functionally characterized. Among them, GmPT7 (Glyma10g33030.1) showed the highest transcript abundance in nodules (FIG. 7a). Furthermore, the expression of GmPT7 was significantly up-regulated by low P, especially in nodules (FIG. 7b).

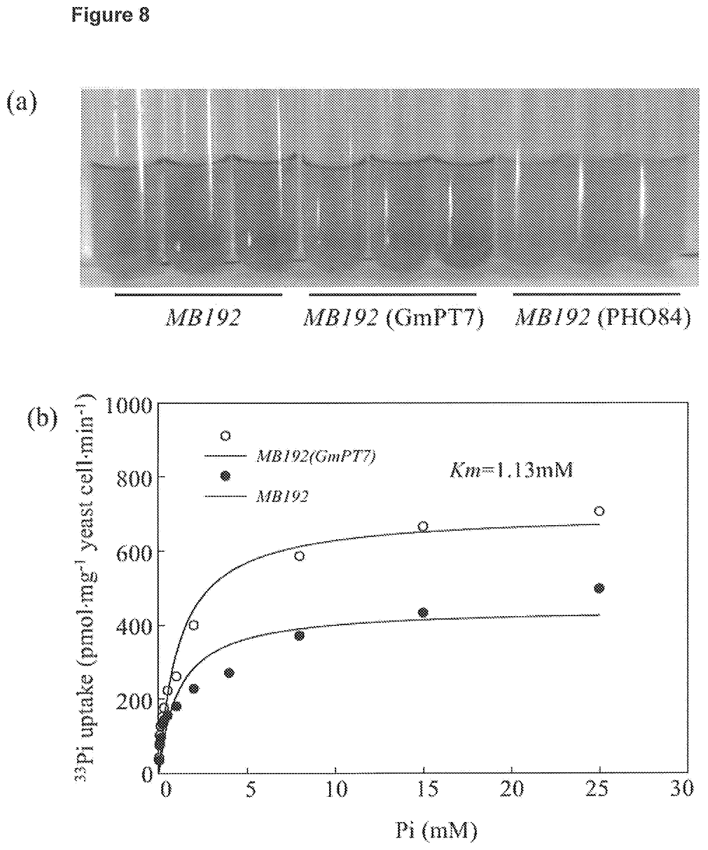

The yeast Pi uptake-defective mutant MB192³⁶ cells harbouring GmPT7 (Yp112-GmPT7) had partially restored their growth in 0.1 mM Pi and grew much better than the empty vector p112A1NE (FIG. 8a). This confirmed that GmPT7 was a high affinity Pi transporter with an mean Km value of 103 μM Pi (FIG. 8b)¹⁷. Interestingly, when Pi was supplied at higher concentrations of up to 30 mM in a ³³Pi labelling experiment; GmPT7 exhibited a low affinity for Pi transport with an apparent mean Km value of 1.13 mM Pi (FIG. 8b). Collectively, these results indicate that GmPT7 might function as a dual affinity Pi transporter in plants.

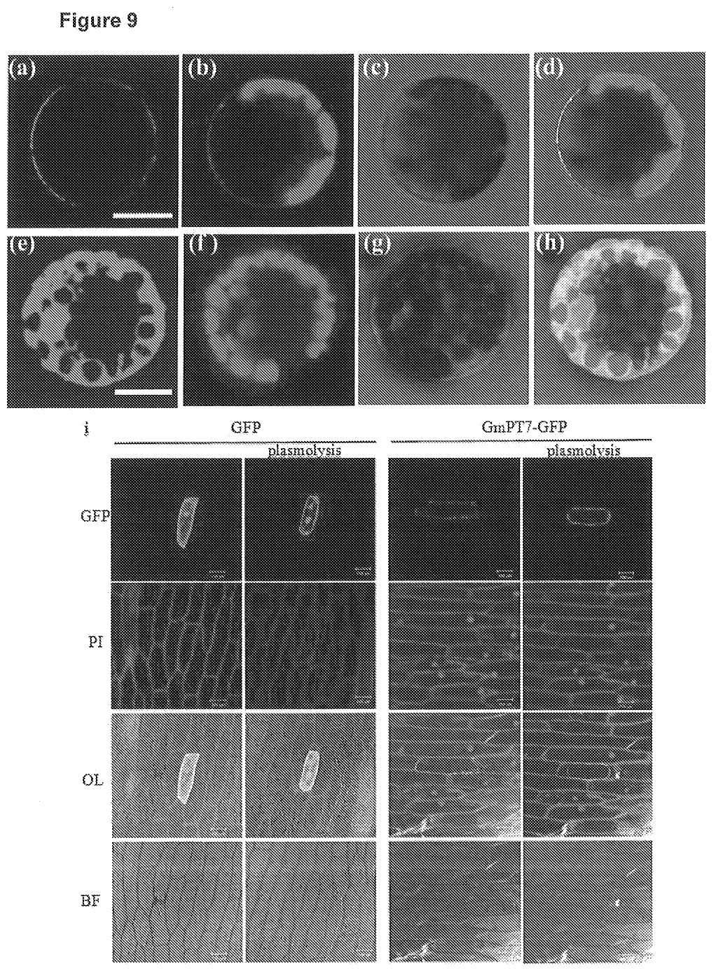

Similar to most plant Pht1 members, GmPT7 was predicted to be localized to the plasma membrane. To confirm its subcellular localization, GmPT7-GFP fusions driven by the CaMV35S promoter were constructed and transfected within an expression vector into Arabidopsis protoplasts and onion epidermal cells. As a result, the fused protein was restricted to the plasma membrane (FIG. 9), indicating that GmPT7 is a plasma membrane-localized protein.

Tissue Localization of GmPT7.

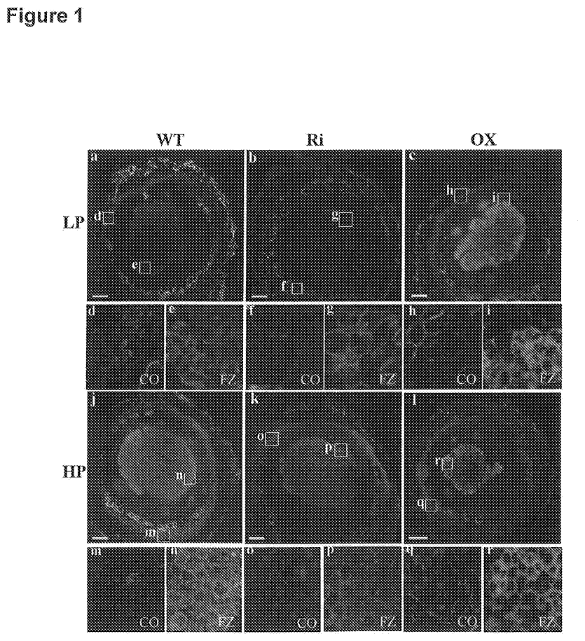

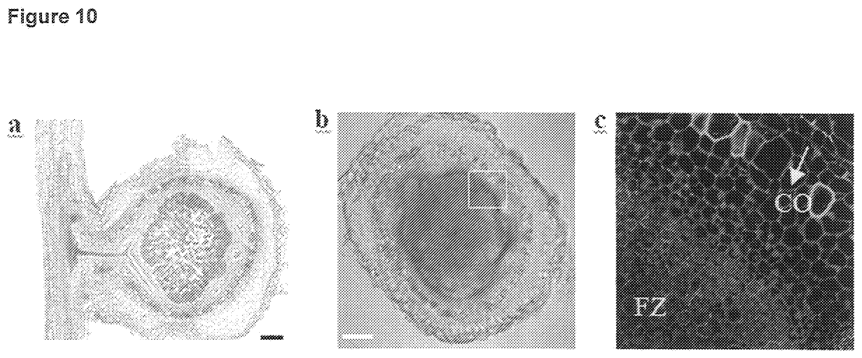

To observe the tissue specificity of GmPT7, GUS staining was performed in transgenic plants carrying the putative promoter region of GmPT7 fused to the β-glucuronidase (GUS) reporter gene (proGmPT7::GUS). The results showed that GmPT7 was localized to the symbiosomes and nodule cortex (FIG. 10a). Furthermore, the result was confirmed with immunostaining using the GmPT7 antibody in soybean nodules (FIG. 1, FIG. 10b, c). These results suggest that GmPT7 is involved in Pi uptake by nodules and Pi translocation between symbionts.

Altered Expression of GmPT7 Changes Pi Uptake and Translocation in Symbionts.

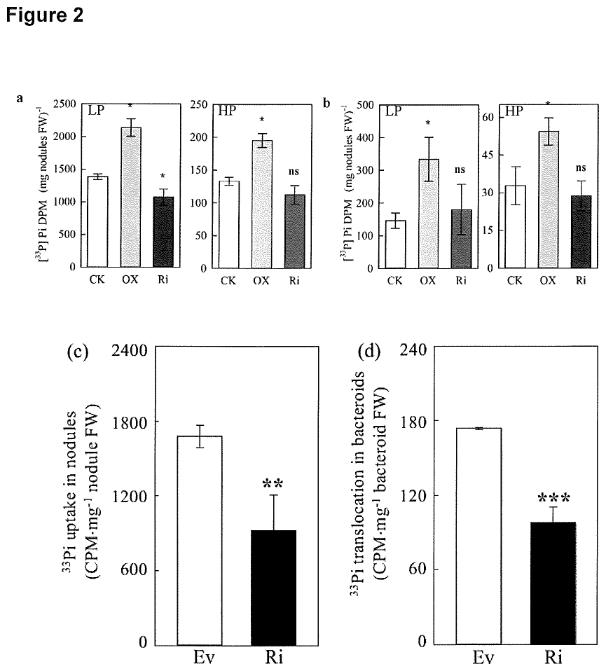

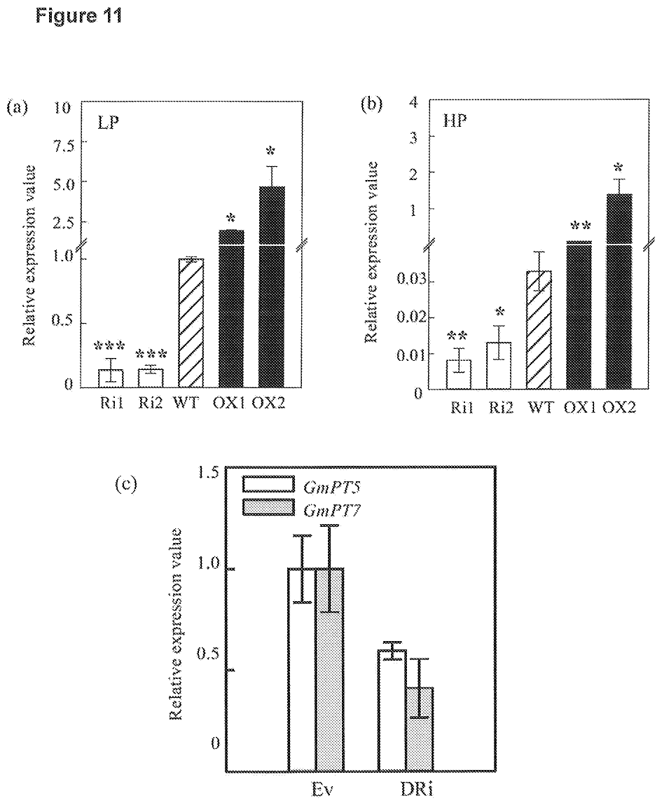

The physiological roles of GmPT7 in nodules was investigated using GmPT7 overexpression (OX) and knockdown (Ri) nodules generated from soybean composite transgenic plants. The corresponding transcripts of GmPT7 in OX and Ri nodules at both low P and high P levels were examined by quantitative real-time (qRT)-PCR (FIG. 11). In order to determine the roles of GmPT7 on nodule Pi uptake and translocation, direct [³³P] Pi absorption by nodules was evaluated using in vitro assays. Based on the results from [³³P] Pi activities of the whole nodule, we found that nodules could directly take up Pi from the growth medium, and that nodules pre-grown in low P absorbed over 10 fold more [³³P] Pi than those pre-grown in high P when supplied with 0.25 μCi of H₃³³PO₄ (FIG. 2a). Compared with control lines (CK), GmPT7-OX nodules absorbed 55% and 46% more [³³P] Pi, and GmPT7-Ri nodules absorbed 22% and 21% less [³³P] Pi at low P and high P levels, respectively (FIG. 2a).

We also detected [³³P] Pi activities of symbiosomes isolated from nodules. The [³³P] Pi activities in the symbiosomes of GmPT7-OX transgenic nodules were 129% and 66% greater than in CK lines under low P and high P conditions, respectively. However, the [³³P] Pi activities in the symbiosomes of GmPT7-Ri nodules were not significantly altered compared to the control (FIG. 2b). The above results all suggest that GmPT7 plays a critical role in direct Pi uptake by nodules and Pi translocation between symbionts.

GmPT7 Expression Significantly Affects Soybean Nodulation, Growth and Yield.

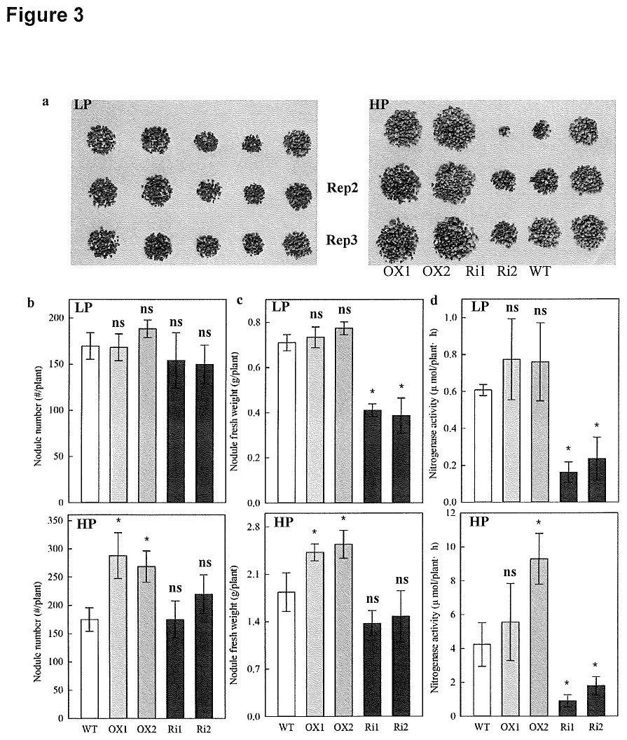

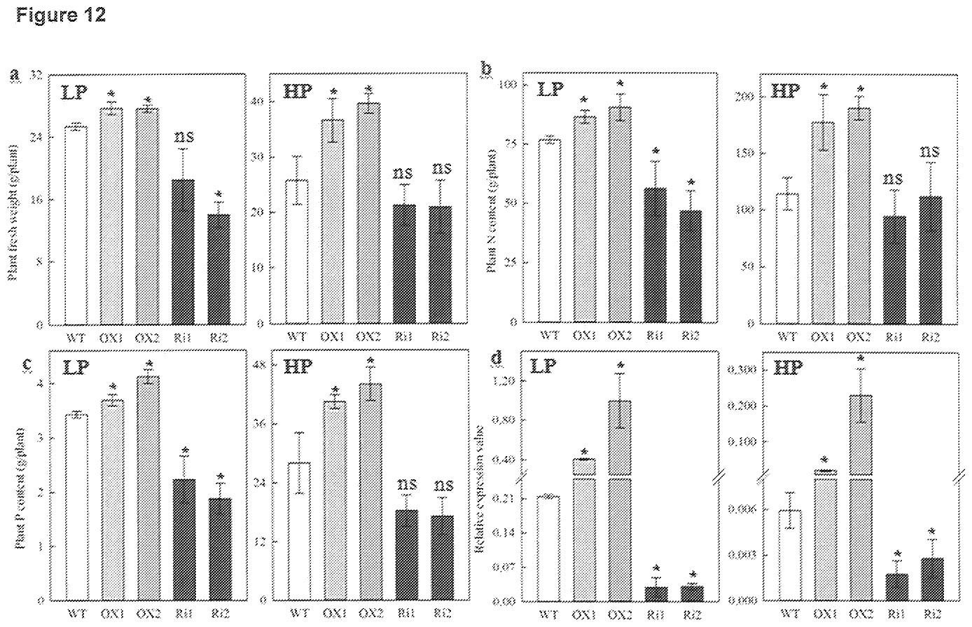

In whole plant transformation lines, the corresponding transcripts of GmPT7 in OX and Ri nodules were examined by quantitative real-time (qRT)-PCR (FIG. 12) and the resulting proteins were determined with immunostaining using the GmPT7 antibody (FIG. 1). The signal in GmPT7-OX nodules was stronger than in WT nodules, while the signal in GmPT7-Ri nodules was weaker. Moreover, the GmPT7 signal in nodules was stronger under low P conditions, which was consistent with the observation of up-regulated GmPT7 expression in low P nodules (FIG. 7b). Alteration of GmPT7 expression resulted in protein variations, which significantly affected soybean nodulation, and subsequently affected plant growth as well as N and P nutrition (FIG. 3, FIG. 12). These effects were also partially dependent on Pi supply. Knockdown of GmPT7 suppressed nodule growth by 42% to 46%, with 61% to 73% reductions in nodule nitrogenase activity at low P. However, overexpression of GmPT7 significantly increased number, fresh weight and nitrogenase activity of nodules in high P compared to WT by 54% to 65%, 32% to 38%, and 31% to 119%, respectively.

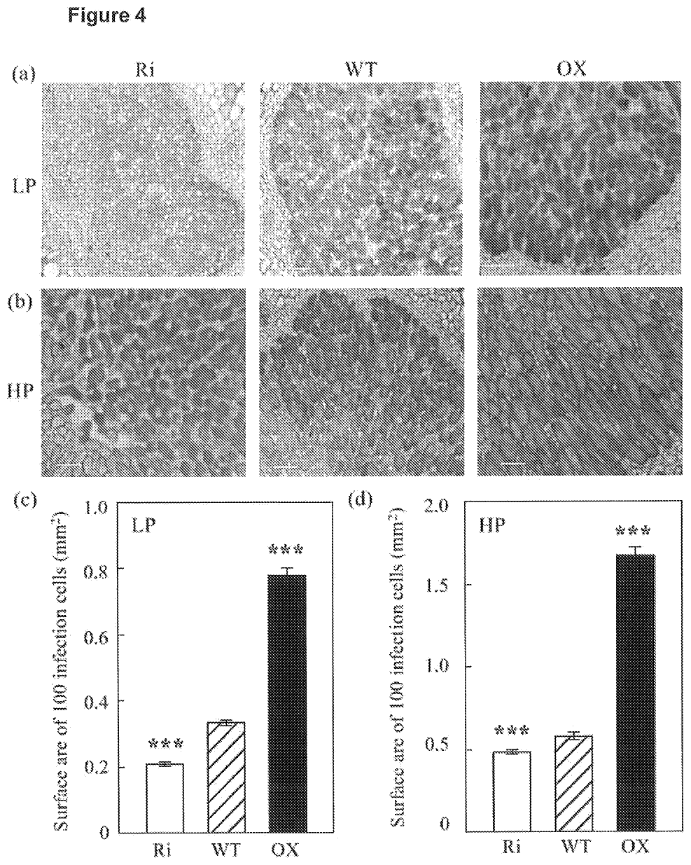

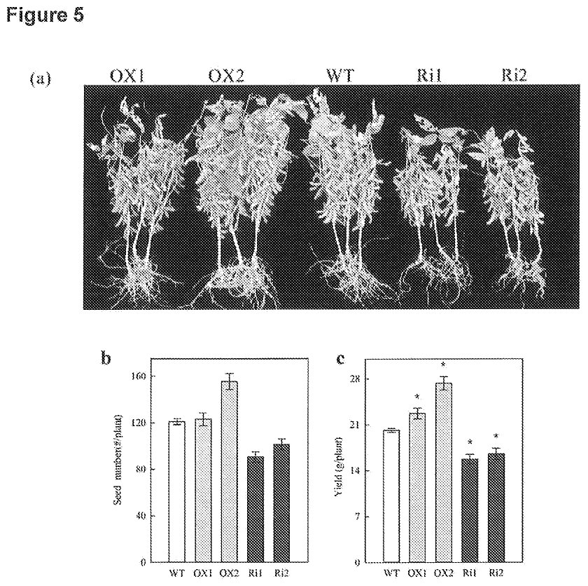

When sectioned, the infected cells are smaller in WT nodules under low P conditions, indicating that Pi starvation severely inhibits nodules organogenesis (FIG. 4a, d). OX nodules displayed larger and more infected cells than in WT plants, while RNAi nodules showed smaller and less infection cells under both low- and sufficient P conditions (FIG. 4), suggesting that GmPT7 is closely involved in soybean nodule development, particularly in bacteroid development. Subsequently, in comparison to observations with WT plants, suppression of GmPT7 inhibited soybean growth as well as N and P content in low P, while overexpression of GmPT7 significantly enhanced soybean fresh weight and N and P content in high P (FIG. 12). As a consequence, in two years of field trails, alteration of GmPT7 expression significantly affected soybean yield as illustrated by increases in seed number of 2%-28% and yield of 13% to 36% for OX relative to WT, and by perspective decreases of 16%-25% and 18% to 24% for Ri lines (FIG. 5).

### Double Suppression of GmPT5 and GmPT7.

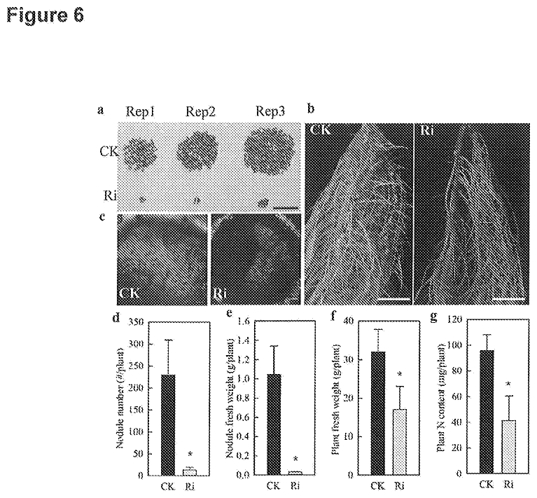

A previously characterized high-affinity Pi transporter, GmPT5, is known to control Pi transport from host roots to nodules in soybean⁶. In the present study, the dual-affinity Pi transporter, GmPT7, played a critical role in direct Pi uptake by nodules and Pi translocation between symbionts. In order to evaluate how much the combination of GmPT5 and GmPT7 contributes to Pi nutrition in nodules, the composite transgenic lines with double suppression of GmPT5 and GmPT7 were generated (FIG. 6). As expected, the combination of GmPT5 and GmPT7 transcript levels significantly affected soybean nodulation under low P conditions. Double suppression of GmPT5 and GmPT7 resulted in nearly complete elimination of nodulation as indicated by 94% and 97% reductions of nodule number and fresh weight compared to CK, respectively, and subsequently inhibited 47% and 57% reductions of soybean biomass and N content (FIG. 6). The nodules that did form in double suppression lines also had very few infected cells indicated by the GFP labelled rhizobia (FIG. 6b), showing the underdevelopment during nodule organogenesis in double suppression lines. These results demonstrate that the combination of GmPT5 and GmPT7 controls most of the Pi uptake and translocation in soybean nodules.

### Discussion

Nitrogen (N) and phosphorus (P) are the two most important mineral nutrients for plant growth, but often are limiting factors for crop production. In geographical areas of low phytoavailability, large amounts of N and P chemical fertilizers are supplied to crops to improve yields². However, overuse of fertilizers in agriculture causes severe environmental pollution, and negatively impacts agricultural sustainability³⁷. As an environmentally friendly N source, BNF by legumes plays a vital role in sustainable agricultural systems³. The process of BNF in legumes is through a symbiotic relationship with bacteroids in nodules²². For protein synthesis and energy consumption, nodule growth and BNF need a large amount of P³⁸. On the other hand, free Pi is released through the BNF process, which may form a feedback loop to inhibit the BNF process, especially under excess Pi supply conditions³⁹. Therefore, it is critical for legumes to control Pi homeostasis in nodules for efficient BNF.

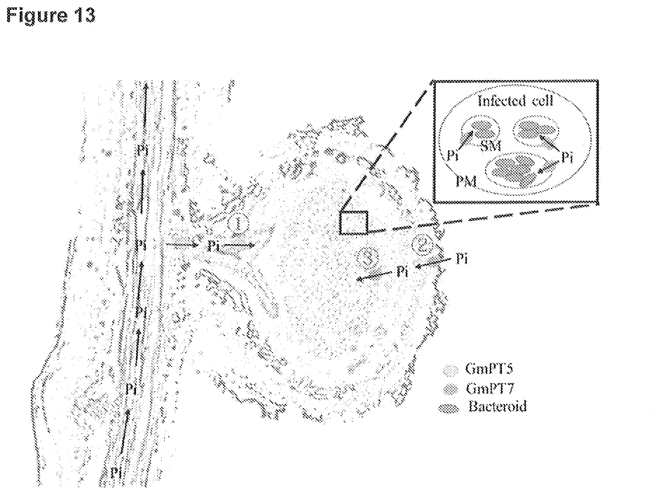

There are three processes involved in Pi transport into and translocation within nodules as summarized in FIG. 13. The first two processes are pathways for Pi entry into nodules, including direct and indirect pathway as previously demonstrated in common bean via a ³²P assay²¹. The step that follows is Pi translocation into bacteroids across the SM for BNF and bacterial requirements. Since Pi transporters are responsible for Pi transport into or translocation within plants⁴⁰, it is reasonable to expect that certain Pi transporters might be involved in Pi uptake and translocation in nodules. The indirect pathway mediated by GmPT5 has been elucidated in soybean nodules⁶. However, mechanisms underlying direct Pi uptake and translocation into bacteroids in legumes has not yet been explored. In the present study, we identified a dual-affinity Pi transporter, GmPT7, which appears to play critical roles in direct Pi uptake by nodules and translocation into bacteroids in soybean with the evidence discussed below.

First, the location of GmPT7 in the cortical cells (FIG. 1, FIG. 10) places it in a location where Pi uptake from the rhizosphere can be controlled, with a notable example being the cortex localized OsPT6, which controls Pi uptake in rice roots¹⁸. Second, Pi concentrations in soils are usually very low (<10 μM)⁴⁰. Since over 80% of Pi fertilizers can be fixed by soil particles, even after Pi fertilization, the soil Pi concentration might still be lower than 100 μM in soil solution⁴¹. Therefore, the Pi uptake process from the rhizosphere often requires the involvement of high-affinity Pi transporter⁴⁰. A recent proteomic study on soybean symbiosomes identified many new symbiotic proteins, of which two are Pi transporters, GmPT5 and GmPT7²³. GmPT5 has been demonstrated to not function in direct Pi uptake by nodules⁶. We have shown that GmPT7 is a candidate high-affinity Pi transporter that might act in direct Pi uptake by nodules. This is further proved by the observation of increased or decreased direct uptake of [³³P] Pi by the overexpression or knockdown of GmPT7 nodules (FIG. 2a). Therefore, GmPT7 and GmPT5 are involved in the direct and indirect Pi entry into soybean nodules, respectively (FIG. 13). In addition, double suppression of GmPT5 and GmPT7 significantly inhibited nodule growth, as evidenced by over 90% reductions in nodule number and fresh weight (FIG. 6), which indicates that the combination of GmPT7 and GmPT5 controls most of the Pi entry into soybean nodules. Above all, our results demonstrated that soybean nodules could acquire Pi not only heterotrophically through GmPT5 from host plants under low P conditions, but also autotrophically through GmPT7 from the rhizosphere under sufficient P conditions.

The SM plays important roles in the exchange of energy and nutrients between bacteroids and host plants³. In a previous study, GmPT5 was weakly localized in the infected cells of nodules, yet overexpression or knockdown of GmPT5 had no significant effects on the accumulation of [³³P] Pi in symbiosomes. In this study, we found that GmPT7 was strongly localized in the infected cells (FIG. 10). Since the concentration of Pi in plant cells is 1000 times higher than that in the soil^{41, 42}, GmPT7 might function as a low-affinity Pi transporter involved in Pi translocation into bacteroids. This is supported by the finding that overexpression of GmPT7 resulted in increased accumulation of [³³P] Pi in symbiosomes (FIG. 2b). Meanwhile, altering GmPT7 expression also significantly affects nodule development, especially infection cell development (FIG. 4), and subsequently affects BNF as indicated by changes in nitrogenase activity (FIG. 3). Collectively, the results herein indicate that GmPT7 also functions in Pi translocation from host cells to bacteroids across the SM (FIG. 13). To the best of our knowledge, this is the first report identifying a functional Pi transporter critically acting in symbionts of legume nodules.

It has been documented that P nutrient status is an important determinant of nodule organogenesis and functionality⁴³. With sufficient P supply, legumes typically nodulate successfully and exhibit superior BNF, as has been noticed for soybean^{6, 44}, common bean⁴⁵, medicagao⁴⁶ and pea⁴⁷. Interestingly, the expression of both GmPT5 and GmPT7 are enhanced by P deficiency¹⁷. Among the 14 GmPTs, GmPT5 transcript abundance was highest in nodules under P-deficient conditions⁶, while GmPT7 transcript abundance was highest in nodules under P-sufficient conditions (FIG. 7a).

Changes in nodulation and growth in transgenic soybean plants with altered transcriptional levels of GmPT5 or GmPT7 are partially dependent on P supply. Under low P conditions, overexpression of GmPT5 or GmPT7 had no significant effects on nodulation, possibly due to a lack of available Pi in the external environment. On the other hand, knockdown of GmPT5 or GmPT7 significantly inhibited nodule organogenesis⁶ (FIG. 3). What's more, double suppression of GmPT5 and GmPT7 resulted in nearly complete elimination of nodule formation and fewer infection cells in nodules (FIG. 6). This shows the importance of Pi entry into nodules as controlled by GmPT5 and GmPT7. In contrast, under sufficient P conditions, overexpression, but not knockdown of GmPT7 significantly changed nodulation and subsequently promoted plant growth and yield (FIG. 3-5), which suggests that GmPT7 can be considered as candidates to improve soybean yield through optimizing nodulation.

In summary, we identified a symbiosome membrane (SM)-localized, dual-affinity Pi transporter, GmPT7, which plays critical roles in direct Pi uptake by nodules and Pi translocation from host cells to bacteroids in soybean. The combination of GmPT5 and GmPT7 controls most of Pi entry into soybean nodules either from host plant roots or from the rhizosphere. To our knowledge, no previous report has described Pi entry into symbionts in legumes. Furthermore, we demonstrate that altering GmPT7 expression impacts grain yield by controlling Pi entry to symbionts and regulating symbiotic N₂ fixation. As Pi is highly required for nodulation in all the legumes, our results suggest that engineering Pi transporters may be useful for increasing grain yield not only in soybean but also in other-legume crops.

### Methods

### Plant Materials and Growth Conditions

For the expression analysis of GmPTs, soybean genotype HN66 and rhizobial strain BXYD3 were used. Seeds were sterilized for 1 min in 3% (v/v) hydrogen peroxide, followed by five rinses in sterile water. Then, sterilized seeds were germinated in silicon sand supplied with ½ strength nutrient solution for 7 days. Seedlings were inoculated with rhizobia for 1 hour and transplanted to low-N (100 μM) nutrient solution at pH 5.8 with two P treatments [composition (in μM): 50 NH₄NO₃, 1200 CaCl₂), 1000 K₂SO₄, 500 MgSO₄.7H₂O, 25 MgCl₂, 2.5 NaB₄O₇.10H₂O, 1.5 MnSO₄.H₂O, 1.5 ZnSO₄.7H₂O, 0.5 CuSO₄.5H₂O, 0.15 (NH₄)₆Mo₇O₂₄.4H₂O, 40 Fe-Na-EDTA, 250 (high P) or 5 (low P) KH₂PO₄]. Nutrient solutions were changed weekly. Thirty days after inoculation, leaves, roots and nodules were separately harvested. Plants were grown in a green house at 21-30° C. under natural sunlight in hydroponics.

### Quantitative RT-PCR Analyses.

The expression of GmPT7 in different tissues under different treatments in soybean plants was determined by quantitative real-time RT-PCR using a Promega SYBR qPCR mix (Promega) on a Rotor-Gene 3000 (Corbett Research, Australia) real-time PCR machine. Specific primers for GmPT genes of the Pht1 family and a soybean housekeeping gene, TefS1 (encodes the elongation factor EF-1a, accession number X56856) were used in this study, which had been published before⁶. The relative expression value was the ratio of the expression value of the target gene to that of TefS1.

Cloning of Full-Length GmPT7 cDNA.

Total RNA was extracted from soybean (Glycine max, genotype HN66) nodules using Trizol reagent following the manufacturer's protocol (Omega Bio-tek) and converted to cDNA using the PrimeScript™RT reagent Kit (TAKARA) reverse transcriptase after treatment with DNase I (TAKARA). Full-length GmPT7 cDNA was amplified using the primers 5′-ATGGCGGGAGGACAACTAGGA-3′ (SEQ ID NO: 9) and 5′-TTAAACTGGAACCGTCCTAGCAG-3′ (SEQ ID NO: 10), which were designed to target the 5′ start codon, and the 3′ termination codon according to the sequence information for GmPT7, which corresponds to GenBank accession: FJ814695.1. The PCR fragment was cloned into the pMD18-T Easy vector (TAKALA) and sequenced by AUGCT methods (http://www.augct.com/ClassID=2).

### Yeast Assay.

The yeast Pi uptake-defective mutant MB192³⁵ and the expression vector in MB192, p112A1NE (abbreviated as Yp112) were used. GmPT7 cDNA was amplified from pMD18-T-GmPT7 using the primers 5′-ATCGGCGGCCGCATGGCGGG AGGACAACTAGGA-3′ (SEQ ID NO: 11) and 5′-ATCGGGATCCTTAAACTGGAACCGTCCTAGCAG-3′ (SEQ ID NO: 12) and cloned into NotI and BamHI sites of Yp112 (Yp112-GmPT7). Yeast strains Yp112-GmPT7 and Yp112 were grown to the logarithmic phase in YNB medium (yeast N base, 6.7 g/L; amino acid mix, 1.98 g/L; Glc monohydrate, 20 g/L; adenine genisulfate, 20 mg/L) and were harvested and washed in Pi-free YNB medium. Then, the YNB liquid media containing 100 μM Pi were used to incubate the yeast strains at 30° C. for 10 h. Bromocresol purple was used as a pH indicator, with a purple to yellow colour shift reflecting the acidification of the liquid medium, which correlated with the growth of the yeast cells. For ³³P uptake experiments in yeast, about 1 mg of fresh yeast cell samples were used following previously described method¹⁸.

### Transient Expression of a GmPT7-GFP Fusion Protein.

GmPT7cDNA was amplified from pMD18-T-GmPT7 using the primers 5′-ATCGTCTAGAGATGGCGGGGGACAA CTAGGA-3′ (SEQ ID NO: 13) and 5′-ATCGGGATCCCGAACTGGAACCGTCCTAGCAGAC-3′ (SEQ ID NO: 14) and cloned into XbaI and BamHI sites of the pBEGFP vector with a cauliflower mosaic virus CaMV35S promoter. The construct and empty vector were expressed in Arabidopsis protoplasts by polyethylene glycol-mediated transformation. In detail, protoplasts were isolated from the leaves of 4-week-old Columbia ecotype Arabidopsis plants using cellulose R10 and macerozyme R10. Approximately 2×10⁵ protoplasts were transformed with 20 mg plasmid DNA via polyethylene glycol 4000 and incubated in a plate under weak light for 12-16 hours prior to observation with a ZEISS LSCM 7DUO (780&7Live) laser scanning confocal microscope (ZEISS, GERMAN).

The construct and empty vector were transiently transformed into onion (Allium cepa) epidermal cells on agar plates by a helium-driven accelerator (PDS/1000; Bio-Rad). In order to eliminate the possibility of cell wall localization, the bombarded epidermal cells were plasmolyzed by adding 30% sucrose solution for 20 min before confocol scanning with red propidium iodide (PI) fluorescence being used as an indicator of cell walls. One day after culturing, GFP expression of target proteins in the bombarded epidermal cells was viewed using a confocal scanning microscope (TCS SP2; Leica) with 488 nm laser light for fluorescence excitation of GFP and detection using a 515- to 545-nm filter (green; GFP fluorescence) and a 610 nm filter (red; propidium iodide fluorescence).

### Histochemical Localization of GUS Expression.

The GmPT7 promoter was amplified from a pMD18-T-GmPT7 promoter construct using the primers 5′-ATCGCTGCAGCCTTGTCTCATGTTACTGCTGC-3′ (SEQ ID NO: 15) and 5′-ATCGCCATGGCCATCA CTCACTAACTCAGCTAC-3′ (SEQ ID NO: 16), and then cloned into XbaI and NcoI sites of pCAMBIA3301 vector to replace the CaMV35S promoter to drive the GUS reporter gene expression. The construct and empty vector were introduced into Agrobacterium rhizogenes strain K599, and then were transformed into soybean cultivar HN66 by hairy-root transformation methods, as described before⁴⁸. Transgenic composite soybeans were grown in a growth chamber with a 16 h: 8 h, light (26° C.): dark (22° C.) photoperiod in hydroponics. Transgenic roots and nodules grown hydroponically were cross-sectioned to a thickness of 50 mm with a microtome (Lecica VT1200S) for GUS staining. Sections of nodules were incubated in sodium phosphate buffer (pH7.0) with X-gluc (5-bromo-4-chloro-3-indolyl glucuronide) overnight at 37° C. Then, the samples were destained with 70% ethanol prior to observation using a light microscope (LEICA DM5000B)⁶.

### Immunostaining of GmPT7.

The synthetic peptide EDKLQHMVESENQKY (positions 269-283 of GmPT7) was used to immunize rabbits to raise polyclonal antibodies against GmPT7. Nodules of 25-day-old grown hydroponically were used for immunostaining using GmPT7 antibody with a 1:300 dilution as described previously⁴⁹. Fluorescence of the secondary antibody (Alexa Fluor 555 goat anti-rabbit IgG; Molecular Probes) was observed using confocal laser scanning microscopy (LSM700; Carl Zeiss).

### Radioactive [³³P] Pi Uptake Assay.

For soybean composite plant transformation, the coding region of GmPT7 was amplified using the primers 5′-ATCGGGATCCTATGGCGGGAGGACAACTAGGA-3′ (SEQ ID NO: 18) and 5′-AT CGACGCGTTTAA ACTGGAACCGTCCTAGCAG-3′ (SEQ ID NO: 19) and cloned into BamHI and M/ul sites of the pYLRNAi vector with a CaMV35S promoter to generate the over-expression construct. Plus, a 423-bp fragment of the GmPT7coding sequence was amplified using the sense orientation primers5′-CATGGGATCCGGCTGCTCTTACCTACTATTGG-3′ (SEQ ID NO: 20) and 5′-CATGAAGCT TAAGGCCACCGTAAACCAGTAC-3′ (SEQ ID NO: 21) and antisense orientation using the primers 5′-CATGCTGCAGAAGGCCACCGTAAACCAGTAC-3′ (SEQ ID NO: 23) and 5′-CATGACGC GTGGCTGCTCTTACCTACTATTGG-3′ (SEQ ID NO: 24) and inserted into the pYLRNAi vector to generate the RNAi construct. Recombinant plasmids were introduced into Agrobacterium rhizogenes strain K599, and then transformed into soybean cultivar HN66 by hairy-root transformation methods, as described before⁴⁸. After removing the main roots, transgenic hairy roots harbouring empty vector (CK), or overexpression (OX), or knockdown (Ri) constructs of GmPT7 were inoculated with rhizobia for 1 hour, and then transplanted into nutrient solution with two P treatments (low P: 5 μM P; high P: 250 μM P) with 500 μM N supply. Fifty days after planting, the nodules from GmPT7-OX and -Ri lines were used for in vitro radioactive [³³P] Pi uptake. After measuring the fresh weight of uniform nodules from different lines, nodules were transferred to 1 mL ³³P labelled nutrient solution containing 0.25 μCi of H₃³³PO₄ for 2 hours, followed by washing with nutrient solution until [³³P] Pi was undetectable in the liquid. Each sample mixed with 4 mL scintillation cocktail was measured by scintillation counting (LS6500 Multi-purpose Scintillation Counter BECKMAN COULTER, USA)⁶.

### Soybean Nodulation Assay.

For soybean whole plant transformation, the coding region of GmPT7 using the primers 5′-GAGCTGAGCTCATGGCGGGAGGACAACT AGGAG-3′ (SEQ ID NO: 24) and 5′-CTAGATCTAGATTAAACTGGAACCGCCTAGCAGA-3′ (SEQ ID NO: 25) was amplified and cloned into SacI and XbaI sites of the pTF101s vector with a CaMV35S promoter to generate the over-expression construct, and a 400-bp fragment of the GmPT7 coding sequence was amplified using the primers 5′-TCAATCTAGAGGCGC GCCGGCTGCTCTTACCTACTATTGG-3′ (SEQ ID NO: 26) and 5′-TGCCGGATCCATTTAAATAAGGC CACCGTAAACCAGTAC-3′ (SEQ ID NO: 27) to generate the RNAi construct prior to cloning into ASCI and SWAI sites, or XbaI and BamHI sites for the sense and antisense orientation of the pFGC5941 vector with a CaMV35S promoter. Recombinant plasmids were introduced into Agrobacterium tumefaciens strains EHA101 and EHA105, which were then transformed into soybean cultivar HN66 as described previously⁵⁰. Transgenic plants were initially screened by leaf painting herbicide assays. Seven-day-old seedlings of WT and transgenic soybean plants were inoculated with rhizobia for 1 hour, and then grown in nutrient solution with two P treatments (low P: 5 μM P; high P: 250 μM P) with 500 μM N supply. Plants were grown in a growth chamber with a 12 h: 12 h, light (26° C.): dark (22° C.) photoperiod for 30 days. Nodules and plants were harvested separately to determine nodule number, fresh weight and nitrogenase activity, as well as plant fresh weight, N and P content. Nodules were separately embedded in paraffin for membrane-enclosed bacteroid observation and then stained with toluidine blue after dewaxing and examined by light microscopy.

### Soybean Yield Evaluation.

The two years' field trials were separately carried out in 2015 and 2016, and the results showed the same tendency. Here, we just presented the results from the year of 2016. For the field experiment, seeds of WT and transgenic soybean plants inoculated with rhizobia were sown on acidic soils at the Ningxi experimental farm of South China Agricultural University (23° 13′N, 113° 81′E) from March to June, 2016. There were four replicates with 15 soybean plants in each replicate for each line. After 15 days, plants were initially screened by leaf painting herbicide assays. At maturation stage, pods were harvested for yield evaluation.

### Double Suppression of GmPT5 and GmPT7.

For soybean composite plant transformation, a 400-bp fragment of the GmPT5 coding sequence was amplified using the sense orientation primers 5′-ATCGAGATCTGAACATGGAGATTCAAGCCGAG-3′ (SEQ ID NO: 28) and 5′-ATCGGAATTCCCAGTGATCAT AGGGAATGGCAA-3′ (SEQ ID NO: 29) and antisense orientation primers 5′-ATCGCTGCAGCCAGTGATCATAG GGAATGGCAA-3′ (SEQ ID NO: 30) and 5′-ATCGTCTAGAGAACATGGAGATTCAAGCCGAG-3′ (SEQ ID NO: 31) and inserted into the pSAT4-35S-RNAi vector, and then a long fragment contained 35S and the 400-bp sense and antisense orientation of GmPT5 cloned into I-SceI sites for the pRCS2-ocs-nptII vector. A 423-bp fragment of the GmPT7 coding sequence were amplified using the sense orientation primers5′-ATCGCCATGGGGCTGCTCTTACCTACTATTGG-3′ (SEQ ID NO: 32) and 5′-ATC GAGATCTAAGGCCACCGTAAACCAGTAC-3′ (SEQ ID NO: 33) and antisense orientation primers 5′-ATCGCTGCAGAAGGCCACCGTAAACCAGTAC-3′ (SEQ ID NO: 34) and 5′-ATCGTCTAGAGGCTGCT CTTACCTACTATTGG-3′ (SEQ ID NO: 35) and inserted into the pSAT6-supP-RNAi vector, and then a long fragment contained supP and the 423 bp sense and antisense orientation of GmPT7 also cloned into PI-PspI sites for the pRCS2-ocs-nptII vector which contained the sense and antisense of GmPT5 fragment. The recombinant plasmid was introduced into Agrobacterium rhizogenes strain K599, which was transformed into soybean cultivar HN66 by hairy-root transformation methods, as described previously⁴⁸. After removing the main roots, transgenic hairy roots harboring empty vector (CK) or double suppression of GmPT5 and GmPT7 (Ri) construct were inoculated with rhizobium strain USDA110 carrying GFP for 1 hour, and then transplanted into nutrient solution under low P treatments (5 μM P) with 500 μM N supply for 30 days. Nodules and plants were harvested separately to determine nodule number and nodule fresh weight, as well as plant fresh weight, N and P content of plants.

### Measurement of Plant N and P Content.

For the measurement of N and P content of plants, approximately 0.1 g of dry samples were digested, and then measured for total N content and total P content using a Continuous Flow Analyzer (SKALAR SAN++, Netherlands)⁷.

### Statistical Analysis

All means and SE values were calculated using Microsoft Excel 2013. Comparisons between groups were performed using Student's t test in Microsoft Excel 2013.

### REFERENCES

1. White, P. J. & Brown, P. H. Plant nutrition for sustainable development and global health. Ann. Bot. 105, 1073-1080 (2010).
2. Dobre, R et al. Influence of N, P chemical fertilizers, row distance and seeding rate on camelina crop. Agrolife Scientific J. 3, 49-53 (2014).
3. Udvardi, M. & Poole, P. S. Transport and metabolism in legume-rhizobia symbioses. Annu. Rev. Plant Biol. 64, 781-805 (2013).
4. Sánchezcalderón, L., Chaconlópez, A., Péreztorres, C. A. & Herreraestrella, L. Phosphorus: plant strategies to cope with its scarcity. Plant Cell Monographs 17, 173-198 (2010).
5. Hernandez, G. et al. Global changes in the transcript and metabolic profiles during symbiotic nitrogen fixation in phosphorus-stressed common bean plants. Plant Physiol. 151, 1221-1238 (2009).
6. Qin, L. et al. The high-affinity phosphate transporter GmPT5 regulates phosphate transport to nodules and nodulation in soybean. Plant Physiol. 159, 1634-1643 (2012).
Li, X., Zhao, J., Tan, Z., Zeng, R. & Liao, H. GmEXPB2, a cell wall β-expansin, affects soybean nodulation through modifying root architecture and promoting nodule formation and development. Plant Physiol. 169, 2640-2653 (2015).
8. Ullrich-Eberius, C. I., Novacky, A., Fischer, E. & Luttge, U. Relationship between energy-dependent phosphate uptake and the electrical membrane potential in lemna gibba G1. Plant Physiol. 67, 797-801 (1981).
9. Daram, R et al. Pht2;1 encodes a low-affinity phosphate transporter from Arabidopsis. Plant Cell 11, 2153-2166 (1999).
10. Nussaume, L. et al. Phosphate import in plants: focus on the PHT1 transporters. Front. Plant. Sci. 2 (2011).
11. Knappe, S., Flugge, U. I. & Fischer, K. Analysis of the plastidic phosphate translocator gene family in Arabidopsis and identification of new phosphate translocator-homologous transporters, classified by their putative substrate-binding site. Plant Physiol. 131, 1178-1190 (2003).
12. Guo, B., lrigoyen, S., Fowler, T. B. & Versaw, W. K. Differential expression and phylogenetic analysis suggest specialization of plastid-localized members of the PHT4 phosphate transporter family for photosynthetic and heterotrophic tissues. Plant Signal. Behav. 3, 784-790 (2008).
13. Poirier, Y. & Bucher, M. Phosphate transport and homeostasis in Arabidopsis. Arabidopsis Book 1, e0024 (2002).
14. Misson, J., Thibaud, M. C., Bechtold, N., Raghothama, K. & Nussaume, L. Transcriptional regulation and functional properties of Arabidopsis Pht1;4, a high affinity transporter contributing greatly to phosphate uptake in phosphate deprived plants. Plant Mol. Biol. 55, 727-741 (2004).
15. Paszkowski, U., Kroken, S., Roux, C. & Briggs, S. P. Rice phosphate transporters include an evolutionarily divergent gene specifically activated in arbuscular mycorrhizal symbiosis. Proc. Natl Acad. Sci. USA 99, 13324-13329 (2002).
16. Nagy, R. et al. Differential regulation of five Pht1 phosphate transporters from maize (Zea mays L.). Plant Biol. 8, 186-197 (2006).
17. Qin, L. et al. Functional characterization of 14 Pht1 family genes in yeast and their expressions in response to nutrient starvation in soybean. PLoS ONE 7, e47726 (2012).
18. Ai, P. et al. Two rice phosphate transporters, OsPht1;2 and OsPht1;6, have different functions and kinetic properties in uptake and translocation. Plant J. 57, 798-809 (2009).
19. Jia, H. et al. The phosphate transporter gene OsPht1;8 is involved in phosphate homeostasis in rice. Plant Physiol. 156, 1164-1175 (2011).
20. Harrison, M. J., Dewbre, G. R. & Liu, J. A phosphate transporter from Medicago truncatula involved in the acquisition of phosphate released by arbuscular mycorrhizal fungi. Plant Cell 14, 2413-2429 (2002).
21. Al-Niemi, T. S., Kahn, M. L. & Mcdermott, T. R. Phosphorus uptake by bean nodules. Plant Soil 198, 71-78 (1998).
22. Oldroyd, G. E. & Downie, J. A. Coordinating nodule morphogenesis with rhizobial infection in legumes. Annu. Rev. Plant Biol. 59, 519-546 (2008).
23. Clarke, V. C. et al. Proteomic analysis of the soybean symbiosome identifies new symbiotic proteins. Mol. Cellular Proteomics Mcp. 14, 46-46 (2015).
24. Benedito, V. A. et al. Genomic inventory and transcriptional analysis of Medicago truncatula transporters. Plant Physiol. 152, 1716-1730 (2009).
25. Colebatch, G. et al. Global changes in transcription orchestrate metabolic differentiation during symbiotic nitrogen fixation in Lotus japonicus. Plant J. 39, 487-512 (2004).
26. Wienkoop, S. & Saalbach, G. Proteome analysis. Novel proteins identified at the peribacteroid membrane from Lotus japonicus root nodules. Plant Physiol. 131, 1080-1090 (2003).
27. Saalbach, G., Erik, P. & Wienkoop, S. Characterisation by proteomics of peribacteroid space and peribacteroid membrane preparations from pea (Pisum sativum) symbiosomes. Proteomics 2, 325-337 (2002).
28. Krylova, V., Andreev, I. M., Zartdinova, R. & Izmailov, S. F. Biochemical characteristics of the Ca²⁺ pumping ATPase in the peribacteroid membrane from broad bean root nodules. Protoplasma 250, 531-538 (2013).
29. Moreau, S., Day, D. A. & Puppo, A. Ferrous iron is transported across the peribacteroid membrane of soybean nodules. Planta 207, 83-87 (1998).
30. Vincill, E. D., Szczyglowski, K. & Roberts, D. M. GmN70 and LjN70. Anion transporters of the symbiosome membrane of nodules with a transport preference for nitrate. Plant Physiol. 137, 1435-1444 (2005).
31. Chiasson, D. M. et al. Soybean SAT1 (Symbiotic Ammonium Transporter 1) encodes a bHLH transcription factor involved in nodule growth and NH₄⁺ transport. Proc. Natl Acad. Sci. USA 111, 4814-4819 (2014).
32. Krusell, L. & Udvardi, M. K. The sulfate transporter SST1 is crucial for symbiotic nitrogen fixation in Lotus japonicus root nodules. Plant Cell 17, 1625-1636 (2005).
33. Moreau, S. et al. GmZIP1 encodes a symbiosis-specific zinc transporter in soybean. J. Biol. Chem. 277, 4738-4746 (2002).
34. David K. A. et al. Acetylene reduction assay for nitrogenase activity: gas chromatographic determination of ethylene per sample in less than one minute. Appl Environ Microbiol. 39, 1078-1080 (1980).
35. Peoples, M. B. et al. The contributions of nitrogen-fixing crop legumes to the productivity of agricultural systems. Symbiosis 48, 1-17 (2009).
36. Bun-Ya, M., Nishimura, M., Harashima, S. & Oshima, Y. The PH084 gene of Saccharomyces cerevisiae encodes an inorganic phosphate transporter. Mol. Cellular Biol. 11, 3229-3238 (1991).
37. Nemery, J. & Gamier, J. Biogeochemistry the fate of phosphorus. Nat. Geosci. 9, 343-344 (2016).
38. Lanje, P. W., Buldeo, A. N., Zade, S. R. & Gulhane, V. G. The effect of Rhizobium and phosphorous solubilizers on nodulation, dry matter, seed protein, oil and yield of soybean. J. Soils Crops 15, 132-135 (2005).
39. Ragsdale, S. W. in The Metal-Driven Biogeochemistry of gaseous compounds in the environment. (eds. M. H. P. Kroneck & S. M. E. Torres) 125-145 (Springer Netherlands, Dordrecht; 2014).
40. Raghothama, K. G. Phosphate acquisition. Annu. Rev. Plant Biol. 50, 665-693 (1999).
41. Raghothama, K. G. & Karthikeyan, A. S. in Root physiology: from gene to function. (eds. H. Lambers & T. D. Colmer) 37-49 (Springer Netherlands,
Dordrecht; 2005).
42. Lopez-Arredondo, D. L., Leyva-Gonzalez, M. A., Gonzalez-Morales, S. I., Lopez-Bucio, J. & Herrera-Estrella, L. Phosphate nutrition: improving low-phosphate tolerance in crops. Annu. Rev. Plant Biol. 65, 95-123 (2014).
43. Vance, C. P. Symbiotic nitrogen fixation and phosphorus acquisition. Plant nutrition in a world of declining renewable resources. Plant Physiol. 127, 390-397 (2001).
44. Qin, L., Jiang, H., Tian, J., Zhao, J. & Liao, H. Rhizobia enhance acquisition of phosphorus from different sources by soybean plants. Plant Soil 349, 25-36 (2011).
45. Drevon, J. J. et al. Phosphorus use efficiency for symbiotic nitrogen fixation in common bean (Phaseolus vulgaris) and its consequence on soil P dynamic. Curr. Plant Sci. Biot. 41, 277-278 (2005).
46. Sulieman, S., Van Ha, C., Schulze, J. & Tran, L. S. P. Growth and nodulation of symbiotic Medicago truncatula at different levels of phosphorus availability. J. Exp. Bot. 64, 2701-2712 (2013).
47. Tariq, M., Hameed, S., Yasmeen, T., Zahid, M. & Zafar, M. Molecular characterization and identification of plant growth promoting endophytic bacteria isolated from the root nodules of pea (Pisum sativum L.). World J. Microb. Biot. 30, 719-725 (2014).
48. Guo, W. et al. A soybean β-expansin gene GmEXPB2 intrinsically involved in root system architecture responses to abiotic stresses. Plant J. 66, 541-552 (2011).
49. Yamaji, N. & Jian, F. M. Spatial distribution and temporal variation of the rice silicon transporter Lsi1. Plant Physiol. 143, 1306-1313 (2007).
50. Wang, X. et al. Overexpressing AtPAP15 enhances phosphorus efficiency in soybean. Plant Physiol. 151, 233-240 (2009).
51. Kochian, L. V., Hoekenga, O. A., Piñeros, M. A., 2004. How do crop plants tolerate acid soils? Mechanisms of aluminum tolerance and phosphorous efficiency. Annu. Rev. Plant Biol. 55, 459-493

SEQUENCE LISTING

SEQ ID NO: 1 GmPT7 nucleic acid sequence (genomic)
ATGGCGGGAGGACAACTAGGAGTGCTTAATGCACTCGATGTGGCAAAGACACAAT
GGTACCACTTCACAGCTATTGTGATTGCTGGAATGGGATTCTTCACCGATGCCTAT
GATCTTTTCTGCATTTCCCTTGTGACCAAGTTGTTGGGGAGGTTATATTACACAGA
CATAAGGAACCCGAAGCCTGGTGTTCTTCCTCCTAATGTTCAAGCTGCTGTGACT
GGTGTTGCACTATGTGGCACTTTAGCCGGCCAACTTTTCTTTGGATGGCTTGGTG
ACAAGTTGGGAAGGAAAAGGGTTTATGGCTTAACTCTTATGCTTATGGTTCTGTGT
TCCATTGCCTCGGGACTCTCTTTTGGCGACACCCCTAAGGGTGTCATGGCCACAC
TTTGTTTCTTCAGATTCTGGCTTGGCTTTGGGATTGGTGGTGACTACCCTCTATCA
GCTACAATTATGTCTGAATATGCCAACAAAAAGACTAGAGGGTCATTCATTGCTGC
GGTGTTTGCAATGCAGGGTTTTGGAATCATGGCTGGTGGAATAGTTGCCTTGATT
GTGTCATCTGCATATGACCACAAGTACGATCTTCCTAGTTACAAGGACAATCCAGC
TGGATCGAAAGTGGATTCGCTTGATTACGTTTGGCGTATCATCTTGATGTTTGGTG
CAGTCCCGGCTGCTCTTACCTACTATTGGCGAATGAAAATGCCAGAGACGGCTCG
CTACACGGCCCTTGTAGCCAAGAATGCAAAACAAGCTGCTTCAGACATGTCTAAG
GTGTTGCAAGTTGAGGTTGAAGCTGAAGAGGACAAGTTGCAGCACATGGTTGAGA
GTGAAAACCAAAAGTATGGCTTGTTCAGCAAGGAATTCGCCAAACGCCACGGGCT
GCACTTGGTTGGAACCACGGTAACTTGGTTCTTGTTGGACATTGCCTTCTACAGCC
AGAACCTTTTCCAAAAGGACATTTTCACTGCCATTGGATGGATTCCTCCTGCACAA
GACATGAATGCAATCCATGAAGTTTATAGGATTGCAAGAGCACAGACACTGATAGC
ATTGTGCAGCACCGTGCCAGGGTACTGGTTTACGGTGGCCTTTATCGATATCATT
GGACGTTTCGCCATCCAATTGATGGGTTTCTTCTTCATGACAGTCTTCATGTTTGC
TCTTGCTATACCTTACAATCACTGGAAGAATCACAACAATATTGGGTTTGTTGTGAT
GTATTCTTTCACCTTTTTCTTCTCCAATTTTGGTCCAAATGCCACCACATTTGTTGT
GCCGGCAGAGATTTTCCCAGCAAGGCTGAGATCTACTTGTCATGGAATCTCAGCT
GCGGCAGGAAAGGCAGGAGCAATTGTTGGTGCATTTGGATTCTTGTATGCTGCAC
AAAGTACGAACCCCAACAAGGTTGATCATGGTTACCCAACTGGTATTGGGGTTAA
GAACTCCCTCATTGTGCTTGGTGTGATCAACTTCTTTGGAATGGTATTCACCTTGT
TAGTACCAGAATCAAAGGGAAAATCACTGGAAGAGTTGAGTGGGGAGAATGAAGA
TGATGGTGCTGAGGCTATAGAGATGGCAGGGTCTGCTAGGACGGTTCCAGTTTAA
SEQ ID NO: 2 GmPT7 nucleic acid sequence (cDNA)
ATGGCGGGAGGACAACTAGGAGTGCTTAATGCACTCGATGTGGCAAAGACACAAT
GGTACCACTTCACAGCTATTGTGATTGCTGGAATGGGATTCTTCACCGATGCCTAT
GATCTTTTCTGCATTTCCCTTGTGACCAAGTTGTTGGGGAGGTTATATTACACAGA
CATAAGGAACCCGAAGCCTGGTGTTCTTCCTCCTAATGTTCAAGCTGCTGTGACT
GGTGTTGCACTATGTGGCACTTTAGCCGGCCAACTTTTCTTTGGATGGCTTGGTG
ACAAGTTGGGAAGGAAAAGGGTTTATGGCTTAACTCTTATGCTTATGGTTCTGTGT
TCCATTGCCTCGGGACTCTCTTTTGGCGACACCCCTAAGGGTGTCATGGCCACAC
TTTGTTTCTTCAGATTCTGGCTTGGCTTTGGGATTGGTGGTGACTACCCTCTATCA
GCTACAATTATGTCTGAATATGCCAACAAAAAGACTAGAGGGTCATTCATTGCTGC
GGTGTTTGCAATGCAGGGTTTTGGAATCATGGCTGGTGGAATAGTTGCCTTGATT
GTGTCATCTGCATATGACCACAAGTACGATCTTCCTAGTTACAAGGACAATCCAGC
TGGATCGAAAGTGGATTCGCTTGATTACGTTTGGCGTATCATCTTGATGTTTGGTG
CAGTCCCGGCTGCTCTTACCTACTATTGGCGAATGAAAATGCCAGAGACGGCTCG
CTACACGGCCCTTGTAGCCAAGAATGCAAAACAAGCTGCTTCAGACATGTCTAAG
GTGTTGCAAGTTGAGGTTGAAGCTGAAGAGGACAAGTTGCAGCACATGGTTGAGA
GTGAAAACCAAAAGTATGGCTTGTTCAGCAAGGAATTCGCCAAACGCCACGGGCT
GCACTTGGTTGGAACCACGGTAACTTGGTTCTTGTTGGACATTGCCTTCTACAGCC
AGAACCTTTTCCAAAAGGACATTTTCACTGCCATTGGATGGATTCCTCCTGCACAA
GACATGAATGCAATCCATGAAGTTTATAGGATTGCAAGAGCACAGACACTGATAGC
ATTGTGCAGCACCGTGCCAGGGTACTGGTTTACGGTGGCCTTTATCGATATCATT
GGACGTTTCGCCATCCAATTGATGGGTTTCTTCTTCATGACAGTCTTCATGTTTGC
TCTTGCTATACCTTACAATCACTGGAAGAATCACAACAATATTGGGTTTGTTGTGAT
GTATTCTTTCACCTTTTTCTTCTCCAATTTTGGTCCAAATGCCACCACATTTGTTGT
GCCGGCAGAGATTTTCCCAGCAAGGCTGAGATCTACTTGTCATGGAATCTCAGCT
GCGGCAGGAAAGGCAGGAGCAATTGTTGGTGCATTTGGATTCTTGTATGCTGCAC
AAAGTACGAACCCCAACAAGGTTGATCATGGTTACCCAACTGGTATTGGGGTTAA
GAACTCCCTCATTGTGCTTGGTGTGATCAACTTCTTTGGAATGGTATTCACCTTGT
TAGTACCAGAATCAAAGGGAAAATCACTGGAAGAGTTGAGTGGGGAGAATGAAGA
TGATGGTGCTGAGGCTATAGAGATGGCAGGGTCTGCTAGGACGGTTCCAGTTTAA
SEQ ID NO: 3 GmPT7 amino acid sequence
MAGGQLGVLNALDVAKTQQYHFTAIVIAGMGFFTDAYDLFCISLVTKLLGRLYYTDIRN
PKPGVLPPNVQAAVTGVALCGTLAGQLFFGWLGDKLGRKRVYGLTLMLMVLCSIASG
LSFGDTPKGVMATLCFFRFWLGFGIGGDYPLSATIMSEYANKKTRGSFIAAVFAMQGF
GIMAGGIVALIVSSAYDHKYDLPSYKDNPAGSKVDSLDYVWRIILMFGAVPAALTYYW
RMKMPETARYTALVAKNAKQAASDMSKVLQVEVEAEEDKLQHMVESENQKYGLFSK
EFAKRHGLHLVGTTVTWFLLDIAFYSQNLFQKDIFTAIGWIPPAQDMNAIHEVYRIARA
QTLIALCSTVPGYWFTVAFIDIIGRFAIQLMGFFFMTVFMFALAIPYNHWKNHNNIGFVV
MYSFTFFFSNFGPNATTFVVPAEIFPARLRSTCHGISAAAGKAGAIVGAFGFLYAAQST
NPNKVDHGYPTGIGVKNSLIVLGVINFFGMVFTLLVPESKGKSLEELSGENEDDGAEAI
EMAGSARTVPV
SEQ ID NO: 4 GmPT7 promoter sequence
TATTTAATTTTAAACAACTCATTAAATGACTTGATTCCCCTATTTCCTCGTTGGTACG
GCAATTAAGATGTACATCTAATCCGGTCGGAACATTAAATAGTATGACCTACGTGT
GTGATTGATTGCCTGCAGTACAATTAACAGTCATAGAAAATCTGTGAAGCCTATAA
GCTTGATGCTTACTCCGTCTAATTTGACTGGAAATATATATAGAAAGAAAAAAAAAA
GGAAATAGAGAGGATAGGGATTGAATAAAAAAATTACTTTCTTAATATTTATTTAAG
AGAAAAAATATTTTATATTTTCTAAGTTATGATAAAATTGGAGATAAATGTTTATTGT
TATATTCTACCACCATTCTATTTATTTAAATAAGTAGAGAAAAAAATATCCACTTATT
TATTTCTTATTTTTATATATTCCAAAAATACATTTTTGTAATACATTTTTATTTTATTTC
TATCACTCCTATTTTCTTCTTCTTATTTTTTTATTTCACTTCTAATTCAAAAAAATATT
ATTGTTTATAATTCCTTTATGTTATTTTTTATCACTTTTTTAAGTTGTAATTTAAAGTT
ATAGAATATAACTTAAGAGATATAATTGGTATTTTTATTCATATTAAAACTAATCAAA
TTTTTAATATATTTTTATATCTTTTATTCATTATTCAAACATTTCATATATTTTTTTTAC
TGCAGTTTCATATAATATTATTATTATTACTATTATTTCTTTTAATAAGTATAAATACA
AGAGTATAATTAATAAAATAATAATTATTACTATCTTGAACTTTTTTTAGAGCATACT
ATTGGATTTTTTTACCTAAAAAATACAAAAAAAAATCCACAAATAATTCTCACGTTTT
TTTTTCCTTAGTTAAGAAATCAGTTCATGGTACATTGAGAAACTTGATTCTCAAGGA
ATCAATTCTCAACATGCTTTTTTTTAATCTAAGAATCGATGTGTTTGATTTTTAAAAA
AAATCTTTTGTTATTTATTTTTAATATTTTGTTTGTATATATTTAAATTTATATATATAT
ATATATATATATATATATATATATATATATATTAGAACTAACAAATGTTTTTTGGCATA
TGTTGTTGTTAGTTTTATATTGATTTTTTTGTTTAAAATTATTTATCATGTTTTCCTTT
CTATGACACAACATGCACCTTGTATCTCCACTATTGTTATGGAAATAAATTTCCAAG
ACTAAGATGATTTTCAATTAATTTATTTAAAAGAAAACACTCATAATTCATTTTTTATC
AAAGAATATTTTTTTAAGAAACAATTATTCATCAAATGAAATATTAAGAATAGGTGAC
ATAAATTGAAAGTAAACATACCTAAAAAAAGAAAAAGAAAAAGATGAAACTAAGTTT
ATCTAGATTGAAGAAGAAAAAAAAAATAAAAAGATAAATAACTCAAAGACTAGATTA
AAGTACATAAAAAAGTTAGAACAACTAATTTATGCAATGACTATACAGATTTATTAC
GCATCAGAGTAATGATCTCAACAACAGTTATTGGTTTTCTTGGTTTTTGCATCAAAA
CAAGGATTTCATTTGGAAAACGCCTAAAAGTTGTATTCAACTCAATTGAACCTTTGG
TACTAAATTCCGAATAAATGAAAAAAAAAATTCTCACATCATTGTCGCATTTGAGCT
CCATATAATTGTATTCAACACAATCATCTATGTAAATTGGTTGACGATAAAAAAGAT
CAAGTAAAATTCTACAACCTCTGTTGACGTCAACAATTATTTTTCTTACGGCATCAA
ACGACATGTTCTCACTATTAGCTTAGTGGTAGAGTGTGTCCCTTTGTATGATGGAA
TCATGTCACCATTACAATACATAACAATGGATGCACTCTCCATGTTTTAAGCAATAT
GGTGAATATCAAGTAAAGGTGTTGAGGTTCATTTAACCCCTACTACTATTTGTATTA
GGTGATAAACTCAAAAGCAGTTTAGATTGTGTCTTGATTTATTATAGTTGGTTTGCG
AATTAAAAAAAAATAGTGTTCAATCAAATCATCTCATAAAAAGTATTGTTCACTCAAA
TTGTCTCGAAAAAAGCATTATGAGGTCAAATCATCTTGATAAAAGCATTGTTCAAAA
AATTCGTCTCAGAAAATGCAATGCTTAATGAATTCGTCTAAGAAAAATGCAGTGCT
CAATCAATAAATACAATGTTCAATGTTCAATCAATTAATAAGAACAAACTGACATTC
ATAGTACACCATAAGTTAACAAGGGTAATTAATGTTGTCTGTGTTCACTAGTTCCAC
ATATGTGAGGTTGCATGTTTCTTGTTGGTTGTCCGCAAACTCGCCCATGTTCTTAT
GATTGAAGTTATTTATTAGGAACAACAGTTTGTTCTTCCAAAGAAATCATTAAAACA
TCATTGATTTTAATCTATCCGAGTTTTGCAACATCTGAAAACCCAACCTAAACCTAC
AAAGATTCAATTCTAGGATCTAGAGAAGACATATCTTGAGCATATGACTGGATTAC
ATCATTTGTAGAAATCAAAATAATAACAACATGACTATTTTTAAATCATAAAATAATT
AATATTCAATTAAACATAACGTATTACATATTTCTCCAGGTCTATGAAGGTGTTGTT
TCCAAAGAAGGTGAATGATTGTAGCTTTCATGGTTTAGGCTTTGGTACTAATGTTA
AAAAGAAGTTAAAGAGTGATATACGAGAGAACGAGAGAAAGTGAGTCGGTGTGCA
TATGATGACATTGCATGCATTGTATCTTGAGTGTTAATTTTGTCAATGGGATCTTGA
TCATTGAACATGTAGTGCTCAAATCGGTGTTATCTAAGCCGATGATTTTTTCGGTG
GGATCTTGGTCATTAAACATGCGTTGCTCTACCCAGTGTTATCTAAATAGTTGATTT
TGACAGTGTGATCTTGGTCATTAAACATGCGTTGATTCAATCAATTTTATCTAAGTC
GTTGATTTTATCAGTGAGATTTTGACCGTTCTTTTTAATGTGCATGTGATAAGTCAT
TAAACATTATAAATATAATATGCAATGAGGTAGTCTAATACATCACCTCAATCAAAA
ACCTTACTGCTACCCTAAACCTAAACCATGACTTCATTATCAATCTCGAAAGTTGTC
TTATCGATTTCATTAACATGAAAACTATGGGAAAGAAACATTATTTGAACCAAAATA
ACTGATACATACAACAACCTAGGTGTCGAAAGTTGTCCTCATGAATTGAAATTTTG
GAATTTTAGTGATTTTTCTTGTTTTTGACCACTTTGTTAATGTGTTTCAAGGTCAATT
GGAGTCCCAAAAGTTGTCTTATTGACTTCATTAACATGAAAACTATGAAAATGCAAC
ATTATTTGAATCAAAATAACTGATACACATAATAATCTAGATGCAAAAAGTTATCCT
CATGGAATGAATTTTTTTAGATAAAAATGATTTTTTCTTGTTATGAAAATTTGCATGC
ACATTTCTGTTAATTAGTTTAATACATGAATATGATAAACAATGAAACCTAACCTAAA
CCCTAATTATGTACCATTTTTACTAAGTATGACCATGAACCCTATCCCTAATTCTGG
ACACCAAACCCTAAATATATTAACTAAACCCTAGGTTTGAAACCTTACCCTAACCAT
AACCCTAATTATGGATACTAAACCCTAAATGTATCAAAGACACATTTGTTTTACATC
AATGCGTTAGGGACACTTTTGATTTAAGCTAGATGAAGGGTGGTGTGACTATGACA
AATTCTAAAAATTGCACATGCCTTGTCTCATGTTACTGCTGCTTGTAAGCATGTTCA
CCATCAGTATAGGAACTACATACATCATGTGTATACATTGAAAAGTGTCTCTAATGT
GTACAGAAGATTGTTTGTTGAATTGCGCAACGAAACATATTGACCACTATCTCATG
AGCCAAGAATCTGCCCCGACCCGAAGATGAAAAGAAATTCTAAAGGTCGTCCTGT
CTCCTCTCGTATACATACCAAAATGGATATCCAAGAATTGGATTAGCCAAAAAGAT
ATTCTATGTGTCGCATCCTAAGCCATTTAAAAAATAAATGTCTTTACCGTGTAGGCT
CACGCCAACAACGTTAAGTTTATGTCATATTGTTTACAATTTAAAAATTTTGTTGTG
AGAAAGAGAAGAAGACAACAAAAGAAAATAGTTTTTTTCTTGGGTCAAACATCGTG
GTTGTCCCTCAACCTATTGTGTTTGATACCAATCTCATTTATTATGTGTGACTAGCT
ACATCAACAAAATTACAAACTAAAATATATGAATAAAGTTTAGAAAAATTTAAAACTA
ACATTTCATCTTATAATTAGTTTTTCATCTCAATCATAAATTATCATATCCCAACAAG
TTATTAGTCTAAGAAAAAAACATAATTGTGAAGGGAGACCACAATAAATATGGGCA
ACCAAGCTTTTTTTTTTTTTTTTACACGGATTAATCACAAAGAAAATTGGTGAATACT
CCACACTAATATCATAGTAGTTAAAAAAATCATAAATTATCATATATGTTAAATTTGT
TGGATTTTGTAATATCTATCATTAATAATAATAATAATAATTTTCATTTAAGAATGAG
AGGGAAAATATTAATTATTAAATTTTATTAAATGATCAAAATTAAAAAAATATTTTTTT
ATTAAATTTTCATTCAAATTTCGTTCCAATATTCTATATTTTTTTATTGTCATGTAAAA
ATATTCTCTGTTTTATATTAATATATACACCAAATTACAAAAATGATTACATACTCTG
TCTGTTTTAACTTTTAATATTTTAAAGCAAAGCAAGCAATTGTTAAAAAGAACGTAAA
GAAATAGATGAAGCAAGCAATGTTATATATATATATATATATATATATATATATATAT
AACAATAGCTTATTCTTTCGGTTTTAACTTTCAATCTTTAATGTCATTAAAAAAAAAA
AACTTATAATCTTTAAAAGCAAGCGGCCAAAGCATTAGAAAGAGCGTAAAGAGATA
GATGAAGCAAGCAAGCGATTTGTGAAAGCGTTGACCAGAAAGTGTGAACTACAAA
GTGTAGGAATGAGCATATACCACCAGATACCTGCGTTGATTCTGGATTCTTGAAGC
CATTCCTTCTATTTTCTGGTGACAGCTTCGTCTTGTGCTATACTATATAACAACTCA
CCCTCTCTCCTTACCTCTTCATTCCCAACACAAAACACTCTCTCCTCCTTCAACCTA
GCCCTGGTCACTACCAACCAAGTGTCCCCCCTCCTCTCCCTTGTTAACAGGTGAG
ACCCTTTTTTCATTTTCCTTAATGGAACTTTTTGGTTATGCATGGCATGGAAGTCAA
ACTAACTCACTGGTAGTTGTAGCCTCTGGTTGAGTTCCTTGGTGGGAGGCCCACT
TTTCTAAGAGCACAAACACAACTTCACTTCCCTTTTCCAATTTTATAATTTACTAATC
ATTACCATTCCACCGCTAAAAATCTGGAACTTGCTTTTTACCCGTATGATATGATAC
GAGGAGACCAATTTCGTGATTCTTCTCCTCTCCTCATCATCCACCACTGACCCACT
CACCCTTTTTGCAAAATAGTGGTTGTGTTATGTTATCCATATTCCATACCCATGAAG
TGAAACACAAAAGAAACAACTTTTAGCCTTACCCATCCTTTTTTTCCTACAGTTTAT
GCTGATTTTCCATGAAACAACTCCAACCCCGTCCCTCTAAACAAAAACCGAACCTT
CTCAACGGAATTAATTGTTCAGGAAATTATTGGCACAAACACAAACCATTCTCCTCT
GCCGTTTGTTTTTTAAATTTATTTTATTTTTATAATTTAGGATGATAAATCTTTTCCAG
TGTCTATGCTTTATTTTGTCTTTTACCAAATTTTGAGTTTTTTCTGGTTTCCTCGTGC
TACCCATGGTCTGCTAGCGACTTGGTTCAACACCTGTAGATTCCCTGTTTGCAGTT
GATTTTCCACACCCTTTTTTTCTTCACGTCTCTGTTCTTTTGGTTTGGTGGGGACAC
CAGAAAAAGAAAAATAGTACCAAATTACCAATGAATTTCCAATTTCTGAACCAAATT
TTCTCTATCATTGTATATACTGAAGTTGACTCATTGGTTGAATCCACACCTTAAGCA
ATTTGGGTGAAGTGGTTGAAAAGTGAGAATAGATCTCACCTTCTTTTACTAAATTCT
CACACGACTTTGATATGAGAAAATTGTGTTGATCTTTCTTTTCATACATAAAATCTT
GTCTATTTAATAATAAACACAATTTTATATGACATGGCTATGTGAAAATATCTATTAC
ATGACAAGCTTTCCGGAGCTGGAATTATAGAGTCGTTGTTAGTTCAGAGTCAAATT
CATATAATATATTAATGTAATAGTTTTATTATAGGAATTTTCAACTTTGGTGGCTGTT
CACTATGAATATATTAGAAAAATACCAAAAAGTAAGGGTTTTTCATCAATCCAATGC
TTCAGCCTGAGAAAGATGAACATCTGCCGTATTTTTTTTTCCTTTCGAACCTTCATT
AAAATTCTTAATTTCTTACAAAAGTCTACTAAAATATACACTTAATTTGTCGATTATT
CAATATTTTATTCTTGATAATTAGTACAAAGTGTGGAACTCGTTATATCATTCCTTCA
CTGTTATTGCAAACACCAGTTCTTTTCCATTCAAATTGTTAGTATTGAAGAGGGCGA
GCCCTGGTGCAGCGGTAAAGTTGTGCCTTGGTGACTTGTTGGTCATGGGTTCGAA
TCCGGAAACAGCCTCTTTGCATATGCAAAGGTAAGGCTGCGTACAACATCCCTCC
CCCATACCTTCGCATAGCGAAGAGCCTCTGGGCAATGGGGTACGAAGGGAAGAA
AAATATGGAATTGCTTGCTTATAGTCTTAGACTATAGTTGAATTTGACACATACAAA
AACTAGTATCTTTTTTTTTATTTTTTTATTTTTGTTTCTTGTGCTGGAAAATTCTAATC
ATTGATTGTGGCACATCAGGTAGCTGAGTTAGTGAGTG
SEQ ID NO: 5 GmPT5 nucleic acid sequence (genomic)
ATGGGGAAGGAGCAAGTTCAGGTGCTGAATGCCCTGGACGTGGCAAAGACACAA
TGGTATCATTTCACAGCAATCATCATTGCTGGAATGGGCTTCTTCACTGATGCTTA
TGATCTGTTCTGTATATCACTAGTCACAAAGCTACTTGGTCGCATTTACTACCACGT
TGATGGGGCTGCAAAGCCTGGTTCATTGCCTCCCAATGTGTCAGCTGCAGTTAAT
GGTGTAGCTTTTGTTGGAACACTTTCAGGGCAACTCTTCTTTGGCTGGCTCGGCG
ACAAAATGGGCCGAAAAAAGGTCTATGGCATGACCCTTGCGCTTATGGTTATAGC
TTCCATTGCTTCGGGTCTATCCTTCGGACACGATGCAAAGACTGTGATGACAACTC
TATGCTTCTTTCGCTTCTGGCTTGGTTTTGGCATTGGTGGAGACTACCCTCTTTCG
GCCACCATAATGTCTGAGTATTCTAATAAGAAGACTCGAGGTGCCTTTATAGCTGC
AGTGTTTGCCATGCAGGGTTTTGGAATTTTGGCAGGAGGTGTGTTTGCTATTATCA
TAGCATCTGTGTTCAAGTCCAAGTTTGATTCTCCACCATACGAGGTTGATCCGTTG
GGTTCGACTGTTCCACAAGCAGACTATGTTTGGAGGATAATTCTCATGTTTGGAGC
AATTCCTGCTGCAATGACTTACTACTCGCGATCCAAGATGCCAGAAACCGCTCGTT
ACACTGCCTTGGTTGCCAAGAATATGGAGAAGGCTGCAGCAGATATGTCTAAGGT
TATGAACATGGAGATTCAAGCCGAGCCAAAGAAGGAGGAGGAGGCACAAGCTAA
ATCATATGGATTGTTCTCCAAGGAGTTCATGAGTCGCCATGGACTGCATCTGCTCG
GAACAACAAGCACATGGTTCTTGCTTGACATTGCATTCTACAGCCAAAATCTTTTC
CAGAAGGATATCTTCAGCGCAATTGGTTGGATTCCTCCGGCAAAAACAATGAATG
CTCTTGAGGAGGTTTTCTTTATTGCAAGGGCTCAAACTCTTATTGCTCTATGCAGT
ACAGTTCCTGGATACTGGTTCACTGTGGCCTTCATTGATAGGATAGGAAGATTCGC
CATCCAATTGATGGGATTCTTCTTTATGACTATCTTCATGTTTGCTCTTGCCATTCC
CTATGATCACTGGACTCTTAGGGAGAACAGAATTGGATTTGTGGTCATTTACTCTC
TCACATTCTTCTTTGCAAACTTTGGGCCTAATGCCACCACATTTGTTGTGCCGGCG
GAGATTTTCCCAGCTAGATTTAGATCCACTTGCCATGGAATATCTTCAGCATCTGG
GAAGCTCGGGGCTATGGTTGGTGCATTCGGGTTTTTATATTTGGCACAGAATCAG
GACCCGAGCAAAGCAGATGCAGGGTACCCTGCAGGTATTGGTGTGAGGAATTCA
CTGCTTGTGTTGGGTGTGATTAACATTTTAGGCTTCATGTTCACTTTCTTGGTGCCT
GAGGCCAAGGGTAGATCCTTGGAGGAGATTTCGGGAGAGCAAGAAGAGGAGACC
AAGGTGTAA
SEQ ID NO: 6 GmPT5 nucleic acid sequence (cDNA)
ATGGGGAAGGAGCAAGTTCAGGTGCTGAATGCCCTGGACGTGGCAAAGACACAA
TGGTATCATTTCACAGCAATCATCATTGCTGGAATGGGCTTCTTCACTGATGCTTA
TGATCTGTTCTGTATATCACTAGTCACAAAGCTACTTGGTCGCATTTACTACCACGT
TGATGGGGCTGCAAAGCCTGGTTCATTGCCTCCCAATGTGTCAGCTGCAGTTAAT
GGTGTAGCTTTTGTTGGAACACTTTCAGGGCAACTCTTCTTTGGCTGGCTCGGCG
ACAAAATGGGCCGAAAAAAGGTCTATGGCATGACCCTTGCGCTTATGGTTATAGC
TTCCATTGCTTCGGGTCTATCCTTCGGACACGATGCAAAGACTGTGATGACAACTC
TATGCTTCTTTCGCTTCTGGCTTGGTTTTGGCATTGGTGGAGACTACCCTCTTTCG
GCCACCATAATGTCTGAGTATTCTAATAAGAAGACTCGAGGTGCCTTTATAGCTGC
AGTGTTTGCCATGCAGGGTTTTGGAATTTTGGCAGGAGGTGTGTTTGCTATTATCA
TAGCATCTGTGTTCAAGTCCAAGTTTGATTCTCCACCATACGAGGTTGATCCGTTG
GGTTCGACTGTTCCACAAGCAGACTATGTTTGGAGGATAATTCTCATGTTTGGAGC
AATTCCTGCTGCAATGACTTACTACTCGCGATCCAAGATGCCAGAAACCGCTCGTT
ACACTGCCTTGGTTGCCAAGAATATGGAGAAGGCTGCAGCAGATATGTCTAAGGT
TATGAACATGGAGATTCAAGCCGAGCCAAAGAAGGAGGAGGAGGCACAAGCTAA
ATCATATGGATTGTTCTCCAAGGAGTTCATGAGTCGCCATGGACTGCATCTGCTCG
GAACAACAAGCACATGGTTCTTGCTTGACATTGCATTCTACAGCCAAAATCTTTTC
CAGAAGGATATCTTCAGCGCAATTGGTTGGATTCCTCCGGCAAAAACAATGAATG
CTCTTGAGGAGGTTTTCTTTATTGCAAGGGCTCAAACTCTTATTGCTCTATGCAGT
ACAGTTCCTGGATACTGGTTCACTGTGGCCTTCATTGATAGGATAGGAAGATTCGC
CATCCAATTGATGGGATTCTTCTTTATGACTATCTTCATGTTTGCTCTTGCCATTCC
CTATGATCACTGGACTCTTAGGGAGAACAGAATTGGATTTGTGGTCATTTACTCTC
TCACATTCTTCTTTGCAAACTTTGGGCCTAATGCCACCACATTTGTTGTGCCGGCG
GAGATTTTCCCAGCTAGATTTAGATCCACTTGCCATGGAATATCTTCAGCATCTGG
GAAGCTCGGGGCTATGGTTGGTGCATTCGGGTTTTTATATTTGGCACAGAATCAG
GACCCGAGCAAAGCAGATGCAGGGTACCCTGCAGGTATTGGTGTGAGGAATTCA
CTGCTTGTGTTGGGTGTGATTAACATTTTAGGCTTCATGTTCACTTTCTTGGTGCCT
GAGGCCAAGGGTAGATCCTTGGAGGAGATTTCGGGAGAGCAAGAAGAGGAGACC
AAGGTGTAA
SEQ ID NO: 7 GmPT5 amino acid sequence
MGKEQVQVLNALDVAKTQWYHFTAIIIAGMGFFTDAYDLFCISLVTKLLGRIYYHVDGA
AKPGSLPPNVSAAVNGVAFVGTLSGQLFFGWLGDKMGRKKVYGMTLAMVIASIASG
LSFGHDAKTVMTTLCFFRFWLGFGIGGDYPLSATIMSEYSNKKTRGAFIAAVFAMQGF
GILAGGVFAIIIASVFKSKFDSPPYEVDPLGSTVPQADYVWRIILMFGAIPAAMTYYSRS
KMPETARYTALVAKNMEKAAADMSKVMNMEIQAEPKKEEEAQAKSYGLFSKEFMSR
HGLHLLGTTSTWFLLDIAFYSQNLFQKDIFSAIGWIPPAKTMNALEEVFFIARAQTLIALC
STVPGYWFTVAFIDRIGRFAIQLMGFFFMTIFMALAIPYDHWTLRENRIGFVVIYSLTFF
FANFGPNATTFVVPAEIFPARFRSTCHGISSASGKLGAMVGAFGFLYLAQNQDPSKAD
AGYPAGIGVRNSLLVLGVINILGFMFTFLVPEAKGRSLEEISGEQEEETKV
SEQ ID NO: 8: ENOD40 promoter nucleic acid sequence
AATCCTTGGTTGGACCTTGTTTGTCAACCCCTGATACGTAATAACCATCATTGATC
ATCAAATTGCATAATCGCGTTGGAAAGTGTTAGCCTATTAAGGCCTAATAAGGCCT
CGTTTGGTCTCAGCCCTCAAATATTGAATTGAATGTTGGTGGATTGCTTCAATTCT
GTGTATGTGTCTATGAATGAACGAAAAATTGGAAAGCTTCCCACTTTAGCTATGAG
GTATCTAAATCTTTGGGCTCTCAATCTCATGCTACCACGGGCCAGTCAAGTGCATC
ATATGCAAGTTCTAATCCATAACCACGACAAAGTCCAAAAGCTATATGGCATAAAC
TAAAATCCATGTACATTTTCAGTCGTAAATGTACATTGTATTTGTATTAGTTGTTACA
TAATTATAAAATAAAAGGTTGTACTCTGTCACTTTTCTATTTAGAGTGTGTTAAGGA
AGTTATTTTAACAAGTTTTTAATTTTTTTTATTAATTGAAAAATTTGTTTGGTTATTTG
ATAATTGATTTTTTTTAATAGTTTCTAGGGTTTTTTGAAACGTTAACTTAAAATATTAT
TTTTTGAAATTTACTTCAACTAGTGTTTTTTAAAATGTTAATCTCTAATTTTTAGTTTG
TTATATTTATTTTTTTCCTTGAATTTTTATTATATTTCCTTTTCAATCTTTTCTATTTAA
GGTAAAATTTTATTGTTATTTTTTCTTTATGTAATTTTATATTTTCTAACTATTTCAAT
AAATAATATTATCAAACACTTATGATTCAATAAGATAATTTTTTAATTTTCAATTAACT
TTTAGCTATCGGTTAGCTTTTCAACTTTCAAGTAATTTTTCATGTAGTTCTATCAAAT
ATAGCATTAATGTATTGCTTTTTCTTATTGAAAAATAATAAAATAATTATTTTAAGGC
TTTTTAATTACTTTATTTATGATAAAAAAATTCAAACAAGTGTTTTAATTAAAAATTAT
CTTAACAAAACACTAAATGGGTCTTAATAAGTTGTTGTAGAAATTTAACTATTTTAAA
TAATCATTTTTTTTCTTGTGTATAATGATACTACTAGTATTTTAGTTACAACTTCAAC
TTTATGTAAACTACTCAAATCTCTCACTACTAAGTTGATCCTAGTATGTTTTAATTAC
TTTCTTCTATTGATGAATGAACTGTAATACGAGTGTTGACATATAATTTTGATTTTGA
TTTAAATTCAAAATCATTAGAATTACGAGTTTGTTTTTCTTAGCTAATTATATTTCATT
AGACTAAAATATAAATTATTATAATTTTAGATTTTCATTCTCGCATATTATTTTCAGTT
ATATATATAACAAAAAAATGCTTAATTTATTAAGAACAATAAAAATAGTTAATTTTAAT
TAATATCATCATAAATTTAAATTTTATTTCAAAAATACTCATAAAATTTATATTTTTAA
TAACTCAATAAATGTATTCACATACATTCATGGATAATGAGTGATATGTAATTAGTA
GACTTCACATTGAATCAATCATATAAAAAGAAACTATCAATTAATATATTTAAAAGTA
ATAATATATTTCTTATAACTAGGATATTTTTTTATAATTTATTTCTTATACTACATTTAT
TACATATGAAGCAGAAAAAAATCATTTATTATACAAAATTTCAAATTTAATAAAACTA
TTGACTATAAAAAGGTTTTTTCTTTAATAATTCTAGACAACAATTAATTCAATAAATT
TCAAGCATTAATATTACTTTTAATTTATGCATAATTTTTCTATAATTTTAAATTTATGC
ATTTTATTATTATCTTTATCCATAATATCATTATTAAAAAAATTATTATTAAATATAATA
AAAAATACTTATTATTATTATACAAGATGATTAAAATAAAAGAAAAGGGAAAATAGA
AGAAGGATCTAAATACCATAGATTAAAATACATAAATTTCAAAGTTTAAAAAGAAAA
TAAAAAGGAAGATCAGTTTAATTGGGTTGGAAAAAGAAAAATATGAGAACCAAATT
CATTGCAATTAATTTGAGTTTGAAGTAAAATAAACTAAATAATTCAATCAATTCAAAT
TGATTTAGTTTAAATTATTATCTTGATTAAGTGAATTAAATCCATAAACACCTTAAAG
TGCATTATAAAAATCGTCACTAAGGCACTAAACTTTTTTATTTTCTTGATTTGCCAG
ATTTCCAATGTAGAGAAGACGGACTTGTTAAGAAAAATATGCCTTTTTCTTCTGAGA
TTCATTCGATTTTTACCGTAATGGTATCCACGTTCTTAAGGAAAAGAGTTGAAATTG
AAACTCTATGAGAAGCAACCAAGGTGTGGTACCCTCCGTGCATACTTTGGGATTG
TTACCTTAACTTAAATGCACGTTCTAATTGTGTAATAATTAGACAAGCGTCTTAACT
TAAATGCACGTTCTAATTGTGTAATAATTAGACAAGCTTACTTGTTACATTTATTTTA
ACATTTTGATATAAATCTTCTTATATTAGAGAGATTTTTTATTTTCTCTAGTAACCGA
AACCAAATATGTCCCGTTTATTGTTGTATCATTTTGAGTTTAATATAATTTAAATTTT
TCTCAAAAAATTAACATGCCTTCCAAAATTTGACTGTAACTTTTATTTAACTTATATA
ACAAGTTTCCAACTGGCAACTACACTCAAAAGCAAAAGACTTTCTCGAAATTTCTG
GGTGTCCAAAACCGAAGATGAATGGCTGGTTTTGGAGAGGTGCTACTAAACCCGA
TATACTTTTTTTTAATCATGATCTTAAAGATATAGAGATTAAAACATACGAAATACAT
CATTTTAATTACTAAAAATAATAAATATTAAATGACATTTACATTTTTCATACAATAAA
TGCGTCTTTCTTTAGTAATTACTTTCTACTTTTGTTAACATCTGTTTTTTTTTTCTTTT
AATAAATAAGATTGTTATTGATGAAGGTTCGCAATTCAAAACTCAAGTGCCTCATCT
ACTAACTAACATAAAGCGATATAATCCGATTGATAAATAATTATGTTGCTTAAATATT
AGAAATTTTATTTCTGAGTCGATGTATATAAAAAAATATTTATTAGAAGATATTAATC
TCATAAATAAATTTTCATATATTAAAAGATTAATTTTTTACTTTCAATTAGTAATCGAC
TTGGTATAAAAAAAAAAGCCTAATTCAGGTTCATCGCTGTTACTCTTTTTAAAATCT
CTATTTTGCTCTTTTTGAGATCTTAGGGGTTACAGATACCTGCAAATGACTTTCAGA
AATTTGGGAATCTTCTCTTTACCACATGTAATATATGAAGGGCCATTTAAGAAAAGG
TATAAATGTTATATTATAAGATAGGGGCAACCAAGATTATGGCAATATAGATTTGGC
ACTGTTGGAAATTGGAATAGTGTCACCTCCTAAATAAAGCTGGAGTAAAGAATGTT
GAAGGCTACCTAGTACCTGCAATGGTTTATCTTTGACCAAAGATCTAGTGGGGGTT
GCCACATATAAGAATTGTAGAAGAATATCACACCCGGAATATACAAGTACACGGTA
AAGTAGTCTTGATCTTGGACCGAAAATAATGAGAACAAGGGCCCCATAAGTTTGAT
TCCAAATTGAAACCACATCATGGAAAAGTCACTTGTCCTACAACTTCCCATGCCAT
GTGGAAAGTGGATGGCAACATTTATAATTACTACGATGATGACTGCTTGGTGAGTC
ATCCAAGTTTGAATGTAAATTGAACTGAAGTCAGTAAAATTTGGAAATTTTGGATTA
TAAGGGACCCAAAACTTGATTACAAATTGGAGCCAACTTTGAGATACTTACATTTTC
CGTTGGTCGTGGTTTGTCCATTTGAAAGTGGTGGGCAATAATGATAGTTTCTATTA
TGATTTTTTAGTAAAAGTTGTAGAATCGGGAGACACTTCTAATACTGCTAGATAGG
GTTAATGACATACTGAAACTATCAACAAATACCACTTAAAATATTACTGCATTATGC
AAAGATTGAAACCATAAACAGTTCCACCTGGTTGTAGTTTATATTCTTTTATGATCT
ATGAAGAAAACAACAAGGATCCAACTTACTTTTCAGCAAAAATAGAATTCATTTCTC
ATAATTTAGTTCTTCCCTTTATCTTATTGACTAATTTGATTTTTAACCCTTCTTTGACT
AGGTTAATTAAATGGGTTTGTTGTTTCATACCAATGGCCACAAAGAAAAGTTCATAA
AACAAGCTGTGAATTGTGATCTCATCCAGTGTAATATAATAGGCAATGCTGATGAC
ATGTACAAACAATAAGTGGCTACCAACAGTAATAAATAAACAGCATAGAGAGATTC
CAAAAGGGGACTTAAAAGCCATAAAAAATATTTTGTTTTATTGCTTGAAGAAGAGG
CAAACCAAAAGCTTGAATGGATTGTCTTGCTGCTATGATTGTGTGCATATCTGAGA
AGGAGATCACTGCCATGTTTAGTAAAATTCATTAAAGAGTGTTAGAGGAATTTTGAT
TAATCTATAAAGCCGGCTAGGGAAAGAAAAATGACTGTTGGATATAAGTTCAAGCT
ATTGTATTGACTGCTACTTTTTTTTTTTAATATATAAAATAGAGAGCTTCACTTCTTG
ATGATGTGATGACTAATCACTCTTTAATTTCAAAAGGAAAGTTGAATTGGCCCCTA
GATATTATGAAAAGAAGACACATGATAGGGATCAATTAACAACTAAGCTAAACAGT
ACAGTATGGCTACATACGCGGCCCAGATTATTTTTAGCTCTGAAATCAAAATCATG
GTATTTTTTGAAACATTATGATATATAATTAAAAAGAAGAAGACAAAGTGGTTTGCT
GGCATGGCTGGTGAGAGAGAACGAAATTAGTGGAGAGTAAGGAATTTAAAATAAT
TTATCATCGATGTTAAATTCTTTTAGCAAATCCTCGTAACATATTTCTAATTATTTCA
AACAGAAAGTGTAAACGTGTTTAATATCTTGGAGAGGGTTATTTAATTGGTTCATTA
AGAATATATAAAACTATTTTTGAAAGCAAAGTGGGCTTTGAGTCTCAGCAAGTGGA
TTCACTGCTGCAATAAGAGCCTCCTTCTATCAAAAGTCAAGCACGAATGAAAAAGT
AAAGTGTGGGTGCCCATGTTTTAAAGTTTATATCGACACTTTGACATTCATATCCTT
TGTATTTCAATTAGATTAGATATGTTCTACGGACATTATTTGATCTTTAAATTCCCTC
AAATGCCTGTGGTGAGCTAATATAATATGTAGTGTAGTGTATGTATGTATGCGGTA
AAAAAATAAGCTGAAATTTGGCGAGGACAATATACAAGTCCCAAATTTAATATGAAT
ATATTATAAACTAAGTACTGCTTAAATAGTAGAAAAACAGAATGAGATAATCAAATT
AGGTCAGGGATCTCAAAACTTCATTCCCATGTAAACCAGTAGAAGAAAAATAAGTG
TGTAAGATACAATTAGGTCAGGTCTATGCTGGTTAGAGTTAGATTTAAGATTCAAG
AAAAAGTTAGGCCGGTAATTAGGTTAAGGTTTAGATTCAATAAACCAAATAAATGTT
TTTTTTAATCCATTTAATTATTTTAGTGAGCACAGTACAAATTTCTTTCTTGAGTTTC
AACTTTTGATGGTTTTTTAAAATGAAAAAGTAATAAGCAAATGGATAATGAAAAGAT
GATGATAGCACTTCTTAGTTCTCAATCATCAACTATTTAAAACAATGGTCAGAGGCT
AACTTCTCCACTAGTTTTTCTGTGTAGAGCCCTTTGGACACACCCTCTAAACCAAT
CTATCAAGTCCTGAATCTGGTGAGCAAATATGGAGCTTTGTTGGCAAACATCCATC
CATGGTTCTTGAAGAAGC

1. A method of increasing at least one of yield, nodulation, nitrogenase activity, the rate of biological nitrogen fixation, nitrogen content and phosphorous content in a plant the method comprising increasing the expression of a nucleic acid sequence encoding a phosphate transporter (PT7) polypeptide.

2. (canceled)

3. The method of claim 1, wherein said increase in yield is an increase in seed yield, preferably an increase in seed number.

4. (canceled)

5. (canceled)

6. (canceled)

7. The method of claim 1, wherein said method comprises introducing and expressing in said plant a nucleic acid construct comprising a nucleic acid as defined in SEQ ID NO: 1 or 2 or a homolog or functional variant thereof.

8. The method of claim 7, wherein said nucleic acid is operably linked to a regulatory sequence, and wherein the regulatory sequence is selected from a constitutively active promoter and a nodule-specific promoter.

9. The method of claim 1, wherein said nucleic acid construct further comprises a nucleic acid sequence encoding a PT5 polypeptide.

10. The method of claim 1, wherein the method comprises introducing a mutation into the plant genome, wherein said mutation is the insertion of at least one or more additional copy of a nucleic acid encoding a PT7 polypeptide or a homolog or variant thereof such that said sequence is operably linked to a regulatory sequence, and wherein such mutation is introduced using targeted genome editing.

11. (canceled)

12. (canceled)

13. The method of claim 7, wherein said homolog or variant has at least 80% sequence identity to the sequence represented by SEQ ID NO: 1 or 2.

14. The method of claim 1, wherein the expression of a nucleic acid encoding a PT7 polypeptide is increased relative to a control or wild-type plant.

15. The method of claim 1, wherein said plant is a legume.

16. The method of claim 15, wherein said legume is soybean.

17. A plant obtained or obtainable by the method defined in claim 1.

18. A plant wherein the expression of a nucleic acid encoding a PT7 polypeptide is increased in at least one root nodule compared to the level of expression in a control or wild-type plant.

19. The plant of claim 18, wherein said plant expresses a nucleic acid construct comprising a nucleic acid as defined in SEQ ID NO: 1 or 2 or a homolog or functional variant thereof, wherein preferably said construct is operably linked to a regulatory sequence.

20. The plant of claim 18, wherein the plant carries a mutation in its genome wherein said mutation is the insertion of at least one or more additional copy of a nucleic acid encoding a PT7 polypeptide or a homolog or a variant thereof such that said sequence is operably linked to a regulatory sequence.

21. (canceled)

22. (canceled)

23. (canceled)

24. The plant of claim 1, wherein the plant is a legume.

25. The plant of claim 24, wherein the legume is soybean.

26. (canceled)

27. (canceled)

28. (canceled)

29. (canceled)

30. (canceled)

31. (canceled)

32. (canceled)

33. (canceled)

34. (canceled)

35. A seed derived from a plant as defined in claim 18.

36. (canceled)

37. (canceled)

38. (canceled)

39. (canceled)

40. (canceled)

41. (canceled)

42. (canceled)

43. (canceled)

44. (canceled)

45. A method for identifying and/or selecting a plant that has an increase in at least one of yield, nodulation, nitrogenase activity, the rate of biological nitrogen fixation, nitrogen content and phosphorous content, the method comprising screening a population of plants and identifying and/or selecting a plant that has a higher level of PT7 expression than a control plant or a plant from the same or different plant population.
